(19) United States
(12) Patent Application Publication
(10) Pub. No.: US 2025/0206689 A1
(43) Pub. Date: Jun. 26, 2025

(54) CATALYST CONDITIONING AND REACTANT DILUTION METHODS IN PROCESSES FOR PRODUCING trans-1,2-DIFLUOROETHYLENE (HFO-1132E)

(71) Applicant: Honeywell International Inc., Charlotte, NC (US)

(72) Inventors: Akbar Mahdavi-Shakib, Buffalo, NY (US); Haiyou Wang, Amherst, NY (US)

(21) Appl. No.: 18/988,346

(22) Filed: Dec. 19, 2024

Related U.S. Application Data
(60) Provisional application No. 63/613,968, filed on Dec. 22, 2023.
(60) Provisional application No. 63/718,450, filed on Nov. 8, 2024.
(60) Provisional application No. 63/724,859, filed on Nov. 25, 2024.

(51) Int. Cl. C07C 17/25; B01J 21/18; B01J 23/44; B01J 37/08

(52) U.S. Cl. CPC C07C 17/25; B01J 21/18; B01J 23/44; B01J 37/08

(57) ABSTRACT

In a first step reaction for producing 1,1,2-trifluoroethane (HFC-143) from 1,1,2-trichloro-1,2,2-trifluoroethane (CFC-113) as part of an overall method for production of trans-1,2-difluoroethylene (HFO-1132E), several intermediates and/or byproducts are formed, some of which may be considered desired and others undesired. The overall reaction methods and/or specific reactions conditions for producing 1,1,2-trifluoroethane (HFC-143) from 1,1,2-trichloro-1,2,2-trifluoroethane (CFC-113) may be selectively tailored, such as with catalyst conditioning and/or reactant dilution with an inert gas, to usefully convert desired intermediates to the desired product 1,1,2-trifluoroethane (HFC-143) and/or minimize the formation of undesired byproducts.

### CROSS-REFERENCE TO RELATED APPLICATIONS

This application claims the benefit under 35 U.S.C. § 119 (e) of U.S. Provisional Application No. 63/613,968 entitled “CATALYST CONDITIONING AND REACTANT DILUTION METHODS IN PROCESSES FOR PRODUCING trans-1,2-DIFLUOROETHYLENE (HFO-1132E)”, filed on Dec. 22, 2023, Provisional Application No. 63/718,450 entitled “CATALYST CONDITIONING AND REACTANT DILUTION METHODS IN PROCESSES FOR PRODUCING trans-1,2-DIFLUOROETHYLENE (HFO-1132E)”, filed on Nov. 8, 2024, and Provisional Application No. 63/724,859 entitled “CATALYST CONDITIONING AND REACTANT DILUTION METHODS IN PROCESSES FOR PRODUCING trans-1,2-DIFLUOROETHYLENE (HFO-1132E)”, filed on Nov. 25, 2024, the entire disclosures of all three are incorporated by reference in their entireties.

### FIELD

The present disclosure relates generally to methods for producing trans-1,2-difluoroethylene (HFO-1132E) from 1,1,2-trichloro-1,2,2-trifluoroethane (CFC-113), and more specifically to catalyst conditioning and reactant dilution methods for managing the formation of intermediates that may be formed in a first step of an HFO-1132E production process.

### BACKGROUND

1,2-difluoroethylene (HFO-1132) has recently found increased utility for a variety of uses. HFO-1132 may exist as a mixture of two geometric isomers, the E- or trans isomer and the Z- or cis isomer, which may be used separately or together in various proportions. Potential end use applications of HFO-1132 include refrigerants, either used alone or in blends with other components, solvents for organic materials, and as a chemical intermediate in the synthesis of other halogenated hydrocarbon solvents.

Certain intermediates and/or byproducts are produced in the process for manufacturing HFO-1132. It would be desirable to convert any useful intermediates into desired products and/or minimize formation of any undesired byproducts in the process to produce the desired product HFO-1132.

### SUMMARY

HFO-1132 and, in particular, HFO-1132E, is produced from 1,1,2-trichloro-1,2,2-trifluoroethane (CFC-113). In a first step, 1,1,2-trifluoroethane (HFC-143) is produced by hydrogenating 1,1,2-trichloro-1,2,2-trifluoroethane (CFC-113) by reaction with hydrogen in the presence of a catalyst to produce 1,1,2-trifluoroethane (HFC-143). The 1,1,2-trifluoroethane (HFC-143) is then dehydrofluorinated in the presence of a catalyst to produce trans-1,2-difluoroethylene (HFO-1132E) and/or cis-1,2-difluoroethylene (HFO-1132Z). The cis-1,2-difluoroethylene (HFO-1132Z) may then be isomerized to produce trans-1,2-difluoroethylene (HFO-1132E).

It has been found that, in the first step for producing 1,1,2-trifluoroethane (HFC-143) from 1,1,2-trichloro-1,2,2-trifluoroethane (CFC-113), several intermediates and/or byproducts are formed, some of which are considered desired intermediates and others undesired byproducts. The present disclosure is based on the discovery that the overall reaction methods and/or specific reaction conditions of the first step for producing 1,1,2-trifluoroethane (HFC-143) from 1,1,2-trichloro-1,2,2-trifluoroethane (CFC-113) may be selectively tailored, such as with catalyst conditioning and/or reactant dilution with an inert gas, to advantageously convert desired intermediates to the desired product 1,1,2-trifluoroethane (HFC-143) and/or minimize the formation of undesired byproducts.

In one form thereof the present disclosure provides a method for producing 1,1,2-trifluoroethane (HFC-143), comprising: hydrogenating 1,1,2-trichloro-1,2,2-trifluoroethane (CFC-113) by reaction with hydrogen in the presence of a catalyst at a first temperature from at least about 250° C. to at least about 350° C. for at least 5 hours to produce 1,1,2-trifluoroethane (HFC-143); and continuing hydrogenating 1,1,2-trichloro-1,2,2-trifluoroethane (CFC-113) by reaction with hydrogen in the presence of the catalyst at a reduced temperature from about 200° C. to about 275° C. and lower than the first temperature to produce a product composition comprising 1,1,2-trifluoroethane (HFC-143).

In another form thereof, the present disclosure provides a method for producing 1,1,2-trifluoroethane (HFC-143), comprising hydrogenating 1,1,2-trichloro-1,2,2-trifluoroethane (CFC-113) by reaction with hydrogen in the presence of a catalyst and an inert diluent gas at a temperature of at least 250° C. to produce a product composition comprising 1,1,2-trifluoroethane (HFC-143).

### BRIEF DESCRIPTION OF THE DRAWINGS

FIG. 1 is a schematic diagram of an apparatus for the conversion of 1,1,2-trichloro-1,2,2-trifluoroethane (CFC-113) to 1,1,2-trifluoroethane (HFC-143).

FIG. 2 is a graph showing the total selectivity to undesired by-products as a function of time for the 4% Pd/C catalyst at 250±1° C. following pre-conditioning with temperature, as presented in Example 3.

### DETAILED DESCRIPTION

### I. Definitions

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

As used herein, the phrase “within any range encompassing any two of these values as endpoints” or “any range using any two of the foregoing values as endpoints” literally means that any range may be selected from any two of the values listed prior to such phrase regardless of whether the values are in the lower part of the listing or in the higher part of the listing. For example, a pair of values may be selected from two lower values, two higher values, or a lower value and a higher value. For example, a range of as low as 1, 2, or 3, or as high as 8, 9, or 10 followed by this phrase encompasses ranges including 1 to 10, or 2 to 8, or 3 to 9.

In the Examples below, the designation “R” may be used in connection with the various fluorine-containing molecules described herein, for example, “R-143” refers to 1,1,2-trifluoroethane (HFC-143).

As used herein, the phrase “desired product” is 1,1,2-trifluoroethane (HFC-143).

As used herein, the phrase “desired intermediates” include one or more of 1-chloro-1,1,2-trifluoroethane (HCFC-133b), 1-chloro-1,2,2-trifluoroethane (HCFC-133) and 1,2-dichloro-1,1,2-trifluoroethane (HCFC-123a).

As used herein, the phrase “undesired byproducts” include one or more of 1, 1,1-trifluoroethane (HFC-143a), ethane (HC-170), chloroethane (HCC-160), 1,1-difluoroethane (HFC-152a), and HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)).

As used herein, the phrase “based on total moles of organic components of the composition” refers only to carbon-containing components and does not include or encompass non-carbon-containing components such as hydrogen (H₂) or hydrogen chloride (HCl).

As used herein, conversion of a reactant molecule (molecule X) during a reaction is calculated using the following equation:

$$\% conversion of molecule X = ( 100 - molecule X mol . \% in the organic components of a product mixture )$$

As used herein, selectivity to a molecule formed during a reaction (molecule X) is calculated using the following equation:

$$\% selectivity to molecule X = mol . \% of molecule X in the organic components of \\ a product mixture / ( 100 - mol . \% of reactant molecules \\ in the organic components of a product mixture ) \times 100.$$

### II. Overview

The present disclosure provides a method for producing trans-1,2-difluoroethylene (HFO-1132E) from 1,1,2-trichloro-1,2,2-trifluoroethane (CFC-113) according to a three-step process shown below (“Process 1”), which includes the following three steps: (i) hydrogenating 1,1,2-trichloro-1,2,2-trifluoroethane (CFC-113) to produce 1,1,2-trifluoroethane (HFC-143), (ii) dehydrofluorinating 1,1,2-trifluoroethane (HFC-143) to produce a mixture of trans-1,2-difluoroethylene (HFO-1132E) and cis-1,2-difluoroethylene (HFO-1132Z), and (iii) isomerizing cis-1,2-difluoroethylene (HFO-1132Z) to trans-1,2-difluoroethylene (HFO-1132E).

Schematic equations for the three steps of Process 1 are represented below:

### Process 1

(i) CFCl₂→CF₂Cl(CFC-113)+H₂→CFH₂→CF₂H(HFC-143)+HCl
(ii) CFH₂→CF₂H→trans-CFH═CHF(HFO-1132E)+cis-CFH═CFH(HFO-1132Z)+HF
(iii) cis-CFH═CFH(HFO-1132Z)→trans-CFH═CHF(HFO-1132E)

Step (i) may proceed through an intermediate of 1,1,2-trifluoroethene (HFO-1123), wherein 1,1,2-trichloro-1,2,2-trifluoroethane (CFC-113) is first hydrogenated to produce the 1,1,2-trifluoroethene (HFO-1123) as an intermediate, which intermediate is itself then hydrogenated to produce 1,1,2-trifluoroethane (HFC-143).

It has been found that, in Step (i), several intermediates and/or byproducts are formed, some of which may be considered desired intermediates and others undesired byproducts. The present disclosure is based on the discovery that the overall reaction methods and/or specific reaction conditions of step (i) may be selectively tailored, such as with catalyst conditioning and/or reactant dilution with an inert gas, to advantageously convert desired intermediates to the desired product 1,1,2-trifluoroethane (HFC-143) and/or minimize the formation of undesired byproducts.

Further details regarding each of Steps (i), (ii), (iii) are set forth below.

### III. Step (i)

### General Process

The hydrogenation reaction of Step (i) may be carried out in the gas vapor phase in a suitable reactor, for example a tubular reactor made from a material which is resistant to temperature and/or corrosion such as nickel and its alloys, including Hastelloy (for example, Hastelloy C276), Inconel (for example, Inconel 600), Incoloy, and Monel, and the vessels may be lined with fluoropolymers.

The reactor for carrying out the hydrogenation reaction of Step (i) may be first cleaned and flushed with an inert gas such as nitrogen, followed by packing with a catalyst such as those described below. The catalyst may be pretreated within the reactor such as by drying in the manner described further below, followed by metering the reactants into the reactor to initiate the reaction.

The process flow for the hydrogenation reaction of Step (i) may be in the down or up direction through a bed of the catalyst. Products may be flowed through one or more scrubbers to remove by-products from the reaction, such as hydrogen fluoride (HF) and/or hydrogen chloride (HCl), and the reaction products may be collected by capture in a cooled cylinder, for example.

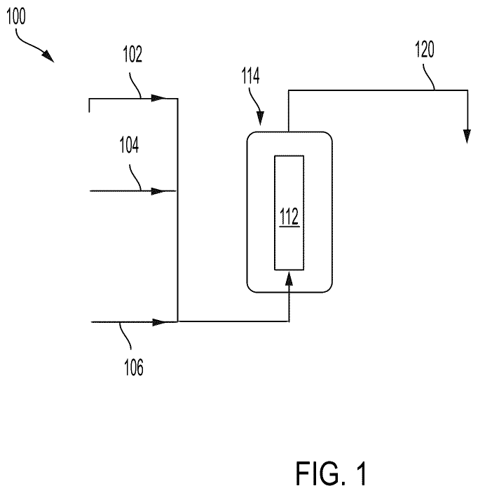

One schematic of a process flow illustrating suitable components for the hydrogenation reaction in step (i) is provided in FIG. 1. Referring to the process flow diagram 100 shown therein, a supply of nitrogen (N₂), which acts as an inert carrier, is provided from stream 102 and a supply of hydrogen (H₂) is provided from stream 104. The organic feedstock which comprises 1,1,2-trichloro-1,2,2-trifluoroethane (CFC-113), is supplied from stream 106. The supply of N₂, H₂, and organic feedstock from streams 102, 104, and 106 are fed into reactor 112 which is surrounded by a box oven 114. The reaction may be monitored by taking samples from outlet 120 and conducting a GC analysis. The catalyst and process conditions used in the reactor 112 play an important role in the Step (i) hydrogenation reaction.

### Catalysts

In the hydrogenation reaction of Step (i), the catalyst may comprise a metal such as palladium, platinum, rhodium, ruthenium, iron, cobalt or nickel. The catalyst active to catalyze the reaction may preferably be palladium metal (Pd), platinum metal (Pt), or a combination of palladium metal and platinum metal.

### Catalyst Support

In the hydrogenation reaction of Step (i), the catalyst may be supported on a suitable support, such as carbon or alumina (aluminum oxide-Al₂O₃). The carbon may be activated carbon. The alumina may be alpha(α) alumina, theta(θ) alumina, delta(δ) alumina, or gamma (γ) alumina. The supported catalyst may be produced by impregnation of any of the suitable supports with a solution of a compound of the desired metal constituent. The support may also be in the form of pellets. After the impregnation step, the solvent may be removed using heat or under vacuum resulting in a solid mass which can be further dried and reduced to form active metal catalyst.

In the hydrogenation reaction of Step (i), the catalyst may be palladium on a carbon support, may be platinum on a carbon support, and/or may be palladium or platinum on an alumina support. The catalyst may be palladium on a carbon support. Alternatively, the catalyst may be palladium on an alpha alumina support.

In the hydrogenation reaction of Step (i), the metal catalysts supported on various catalyst supports are listed in Table 1 below.

TABLE 1
Supported Metal Catalysts - Step (i) Hydrogenation Reaction

| Catalyst | Support |
|---|---|
| Pd | Activated Carbon |
| Pd | alpha(α)-Al₂O₃ |
| Pd | theta(θ)-Al₂O₃ |
| Pd | delta(δ)-Al₂O₃ |
| Pd | gamma(γ)-Al₂O₃ |
| Pt | Activated Carbon |
| Pt | alpha(α)-Al₂O₃ |
| Pt | theta(θ)-Al₂O₃ |
| Pt | delta(δ)-Al₂O₃ |
| Pt | gamma(γ)-Al₂O₃ |

### Catalyst Loading

For each catalyst/support combination (each row) in Table 1 used in the hydrogenation reaction of Step (i), the amount of metal loading on the support is from about 0.01 wt. %, about 0.05 wt. %, about 0.1 wt. %, about 0.2 wt. %, about 0.3 wt. %, about 0.4 wt. %, about 0.5 wt. %, or about 1 wt. % to about 2 wt. %, about 3 wt. %, about 5 wt. %, about 10 wt. %, about 20 wt. %, about 30 wt. %, about 40 wt. %, about 50 wt. % or within any range encompassed by two of the foregoing values as endpoints, for example, from about 0.01 wt. % to about 20 wt. %, from about 0.01 wt. % to about 10 wt. %, from about 0.1 wt. % to about 10 wt. %, 0.2 wt. % to about 10 wt. %, from about 0.5 wt. % to about 5 wt. %, from about 1 wt. % to about 5 wt. %, from about 1 wt. % to about 4 wt. %, or about 2 wt. % to about 4 wt. % based on a total weight of the catalyst and support. For supported noble metal catalysts such as Pd or Pt, the metal loading may be from about 0.01 wt. % to about 5 wt. % based on a total weight of the catalyst and support. Specific examples of additional suitable ranges are set forth below in Table 2.

TABLE 2
Loading for Catalysts (Pd. Pt) on Supports (Activated Carbon,
alpha(α)-Al₂O₃, theta(θ)-Al₂O₃, delta(δ)-Al₂O₃, or
gamma(γ)-Al₂O₃) - Each Catalyst/Support Combination
(Each Row) in Table 1 - Step (i) Hydrogenation Reaction

| From (wt. %) | To (wt. %) |
|---|---|
| 0.01 | 20 |
| 0.01 | 10 |
| 0.01 | 5 |
| 0.01 | 4 |
| 0.5 | 20 |
| 0.5 | 10 |
| 0.5 | 5 |
| 0.5 | 4 |
| 1 | 20 |
| 1 | 10 |
| 1 | 5 |
| 1 | 4 |
| 2 | 20 |
| 2 | 10 |
| 2 | 5 |
| 2 | 4 |
| 0.1 | 0.2 |
| 0.2 | 0.3 |
| 0.3 | 0.4 |
| 0.4 | 0.5 |
| 0.5 | 1 |
| 1 | 2 |

### BET Surface Area

The BET (Brunauer, Emmet, and Teller) analysis is the standard method for determining surface areas from nitrogen adsorption isotherms. The BET surface areas of catalysts may be measured using TriStar II Micromeritics instrument. Catalyst samples are degassed using FlowPrep 060 instrument before BET analysis.

For each catalyst/support combination (each row) in Table 1 used in the hydrogenation reaction of Step (i), the BET surface area of the catalyst may be as low as about 0.5 m²/g, about 1 m²/g, about 3 m²/g, about 5 m²/g, about 10 m²/g, about 15 m²/g, about 20 m²/g 2, about 30 m²/g, about 40 m²/g, about 50 m²/g, about 100 m²/g, about 200 m²/g, or as high as about 250 m²/g, about 300 m²/g, about 400 m²/g, about 500 m²/g, about 600 m²/g, about 700 m²/g m², about 800 m²/g, about 900 m²/g, about 1000 m²/g, about 2000 m²/g, about 3000 m²/g, or within any range encompassed by any of the foregoing values as endpoints, such as from about 0.5 m²/g to about 3000 m²/g, from about 1 m²/g to about 2000 m²/g, from about 1000 m²/g to about 2000 m²/g, from about 0.5 m²/g to about 500 m²/g, from about 0.5 m²/g to about 300 m²/g, from about 0.5 m²/g to about 5 m²/g, or from about 200 m²/g to about 300 m²/g.

For carbon supported metal catalysts (Pd and Pt) used in the hydrogenation reaction of Step (i), the BET surface area may be from about 100 m²/g to about 3000 m²/g, preferably from about 200 m²/g to about 2000 m²/g, more preferably from about 500 m²/g to about 1500 m²/g, and most preferably from about 1000 m²/g to about 1500 m²/g. Specific examples of additional suitable ranges are set forth below in Table 3a.

For alumina supported (alpha(α)-Al₂O₃, theta(θ)-Al₂O₃, delta(δ)-Al₂O₃, or gamma (γ)-Al₂O₃) metal catalysts (Pd and Pt) used in the hydrogenation reaction of Step (i), the BET surface area may be from about 0.5 m²/g to about 500 m²/g, from about 1 m²/g to about 500 m²/g, preferably from about 1 m²/g to about 200 m²/g, more preferably from about 1 m²/g to about 100 m²/g, and most preferably from about 1 m²/g to about 20 m²/g. Specific examples of additional suitable ranges are set forth below in Table 3b.

TABLE 3a
BET Surface Area of Catalysts (Pd, Pt) on Supports (Activated
Carbon) - Step (i) Hydrogenation Reaction

| From (m²/g) | To (m²/g) |
|---|---|
| 100 | 3000 |
| 100 | 2000 |
| 100 | 1500 |
| 200 | 3000 |
| 200 | 2000 |
| 200 | 1500 |
| 500 | 3000 |
| 500 | 2000 |
| 500 | 1500 |
| 1000 | 3000 |
| 1000 | 2000 |
| 1000 | 1500 |
| 100 | 200 |
| 200 | 500 |
| 500 | 1000 |
| 1000 | 1500 |
| 1500 | 2000 |
| 2000 | 3000 |

TABLE 3b
BET Surface Area of Catalysts (Pd, Pt) on
Supports (alpha(α)-Al₂O₃, theta(θ)-Al₂O₃,
delta(δ)-Al₂O₃, or gamma(γ)-Al₂O₃) -
Step (i) Hydrogenation Reaction

| From (m²/g) | To (m²/g) |
|---|---|
| 0.5 | 500 |
| 0.5 | 200 |
| 0.5 | 100 |
| 0.5 | 20 |
| 1 | 500 |
| 1 | 200 |
| 1 | 100 |
| 1 | 20 |
| 0.5 | 1 |
| 1 | 50 |
| 50 | 100 |
| 100 | 200 |
| 200 | 500 |

### Catalyst Pretreatment

For each catalyst/support combination (each row) in Table 1 used in the hydrogenation reaction of Step (i), the catalyst may be pretreated by a variety of methods to improve its performance and effectiveness in the reaction. For example, the catalyst may be dried at elevated temperatures, as low as about 200° C., about 250° C., about 300° C., about 350° C., about 360° C., about 370° C., or as high as about 380° C., about 390° C., about 400° C., about 450° C., about 500° C., about 600° C., about 700° C., or within any range encompassed by two of the foregoing values as endpoints, such as from about 200° C. to about 400° C., from about 200° C. to about 350° C., from about 250° C. to about 350° C., from about 250° C. to about 300° C., or from about 260° C. to about 300° C. Specific examples of additional suitable ranges are set forth below in Table 4.

TABLE 4
Pre-treatment Drying Temperature for Catalysts (Pd and Pt) on
Supports (Activated Carbon, alpha(α)-Al₂O₃, theta(θ)-Al₂O₃,
delta(δ)-Al₂O₃, or gamma(γ)-Al₂O₃) - Each Catalyst/Support
Combination (Each Row) in Table 1 - Step (i) Hydrogenation Reaction

| From (° C.) | To (° C.) |
|---|---|
| 200 | 400 |
| 200 | 350 |
| 200 | 300 |
| 220 | 400 |
| 220 | 350 |
| 220 | 300 |
| 250 | 400 |
| 250 | 350 |
| 250 | 300 |
| 260 | 400 |
| 260 | 350 |
| 260 | 300 |

For each catalyst/support combination (each row) in Table 1 used in the hydrogenation reaction of Step (i), as part of the catalyst pretreatment, the catalyst may be exposed to an inert gas such as N₂. The pretreatment process may take as low as about 1 hour, about 2 hours, about 3 hours, or as high about 4 hours, about 5 hours, about 6 hours, about 10 hours, about 20 hours, or within any range encompassed by two of the foregoing values as endpoints such as about 2 hours to about 4 hours, for example from about 1 hour to about 20 hours, from about 2 hours to about 10 hours, from about 3 hours to about 6 hours, or from about 4 hours to about 5 hours.

### Reaction Conditions-Temperature

For reactions using each catalyst/support combination (each row) in Table 1, the reaction temperature of the hydrogenation reaction of Step (i) may be as low as about 100° C., about 125° C., about 150° C., about 200° C., about 250° C. or as high as about 300° C., about 350° C., about 400° C., or within any range encompassed by two of the foregoing values as endpoints, such as from about 100° C. to about 400° C., or from about 125° C. to about 350° C., from about 150° C. to about 300° C., or from about 200° C. to about 250° C., for example. The temperature may be preferably from about 100° C. to about 350° C., and more preferably from about 200° C. to about 300° C. Specific examples of additional suitable ranges are set forth below in Table 5.

TABLE 5
Reaction Temperature When Using Catalysts (Pd and Pt) on Supports
(Activated Carbon, alpha(α)-Al₂O₃, theta(θ)-Al₂O₃, delta(δ)-Al₂O₃,
or gamma(γ)-Al₂O₃) - Each Catalyst/Support Combination (Each
Row) in Table 1 - Step (i) Hydrogenation Reaction

| From (° C.) | To (° C.) |
|---|---|
| 100 | 400 |
| 100 | 350 |
| 100 | 300 |
| 100 | 250 |
| 100 | 200 |
| 100 | 150 |
| 100 | 125 |
| 125 | 400 |
| 125 | 350 |
| 125 | 300 |
| 125 | 250 |
| 125 | 200 |
| 125 | 150 |
| 150 | 400 |
| 150 | 350 |
| 150 | 300 |
| 150 | 250 |
| 150 | 200 |
| 200 | 400 |
| 200 | 350 |
| 200 | 300 |
| 200 | 250 |
| 250 | 400 |
| 250 | 350 |
| 250 | 300 |
| 300 | 400 |
| 300 | 350 |
| 350 | 400 |

As demonstrated by the Examples herein, the selectivity towards the desired product 1,1,2-trifluoroethane (HFC-143) of the hydrogenation reaction of Step (i) may increase with temperature. However, the overall selectivity to 1,1,2-trifluoroethane (HFC-143) and its associated recyclable intermediates such as 1-chloro-1,1,2-trifluoroethane (HCFC-133b), 1-chloro-1,2,2-trifluoroethane (HCFC-133), 1,2-dichloro-1,1,2-trifluoroethane (HCFC-123a), and trifluoroethylene (HFO-1123) may decrease with high temperature because of increased formation of undesired byproducts such as ethane (HC-170), chloroethane (HCC-160), 1,1-difluoroethane (HFC-152a), HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), and 1,1,1-trifluoroethane (HFC-143a).

### Reaction Condition—Contact Time

For reactions using each catalyst/support combination (each row) in table 1, the contact time of the reactants with the catalyst (Pd and Pt) in the hydrogenation reaction of Step (i) may be as little as about 0.1 second, about 1 second, about 2 seconds, about 5 seconds, about 10 seconds, about 15 seconds or about 20 seconds, or as long as about 25 seconds, about 30 seconds, about 40 seconds, about 50 seconds, about 60 seconds, about 120 seconds, or within any range encompassed by two of the foregoing values as endpoints, such as from about 0.1 seconds to about 120 seconds, from about 1 second to about 60 seconds, from about 5 seconds to about 50 seconds, from about 10 seconds to about 40 seconds, from about 15 seconds to about 30 seconds, or about 20 seconds to about 25 seconds. For example, the contact time may be preferably from about 1 second to about 60 seconds. Specific examples of additional suitable ranges are set forth below in Table 6.

TABLE 6
Contact Time of Reactants with Catalysts (Pd and Pt) on Supports
(Activated Carbon, alpha(α)-Al₂O₃, theta(θ)-Al₂O₃, delta(δ)-Al₂O₃,
or gamma(γ)-Al₂O₃) - Each Catalyst/Support Combination (Each
Row) in Table 1 - Step (i) Hydrogenation Reaction

| From (seconds) | To (seconds) |
|---|---|
| 1 | 120 |
| 1 | 100 |
| 1 | 80 |
| 1 | 60 |
| 1 | 40 |
| 1 | 20 |
| 5 | 120 |
| 5 | 100 |
| 5 | 80 |
| 5 | 60 |
| 5 | 40 |
| 5 | 20 |
| 10 | 120 |
| 10 | 100 |
| 10 | 80 |
| 10 | 60 |
| 10 | 40 |
| 10 | 20 |
| 0.1 | 1 |
| 1 | 2 |
| 2 | 5 |
| 5 | 10 |
| 20 | 120 |
| 20 | 100 |
| 20 | 80 |
| 20 | 60 |
| 20 | 40 |
| 40 | 50 |
| 50 | 60 |
| 60 | 70 |
| 70 | 80 |
| 80 | 100 |
| 100 | 120 |

### Reaction Condition-Pressure

For reactions using each catalyst/support combination (each row) in Table 1, the pressure inside which reactor the hydrogenation reaction of Step (i) takes place may be as little as about 1 psig, about 3 psig, about 5 psig, about 10 psig, about 15 psig, about 20 psig, about 30 psig, about 35 psig or about 40 psig, or as great as about 90 psig, about 100 psig, about 120 psig, about 150 psig, about 200 psig or about 250 psig, about 300 psig, or within any range encompassed by two of the foregoing values as endpoints, such as from about 1 psig to about 300 psig, from about 3 psig to about 250 psig, from about 5 psig to about 200 psig, from about 10 psig to about 150 psig, from about 15 psig to about 120 psig, from about 20 psig to about 100 psig, from about 30 psig to about 90 psig, or from about 35 psig to about 40 psig. For example, the pressure may be preferably from about 10 psig to about 200 psig. Specific examples of additional suitable ranges are set forth below in Table 7.

TABLE 7
Reaction Pressure using Catalysts (Pd and Pt) on Supports
(Activated Carbon, alpha(α)-Al₂O₃, theta(θ)-Al₂O₃, delta(δ)-Al₂O₃,
or gamma(γ)-Al₂O₃) - Each Catalyst/Support Combination
(Each Row) in Table 1 - Step (i) Hydrogenation Reaction

| From (psig) | To (psig) |
|---|---|
| 1 | 300 |
| 1 | 250 |
| 1 | 200 |
| 1 | 150 |
| 1 | 120 |
| 1 | 100 |
| 1 | 90 |
| 1 | 40 |
| 15 | 300 |
| 15 | 250 |
| 15 | 200 |
| 15 | 150 |
| 15 | 120 |
| 15 | 100 |
| 35 | 300 |
| 35 | 250 |
| 35 | 200 |
| 35 | 150 |
| 35 | 120 |
| 35 | 100 |
| 35 | 90 |
| 35 | 40 |
| 100 | 300 |
| 100 | 250 |
| 100 | 200 |
| 100 | 150 |
| 100 | 120 |
| 200 | 300 |
| 200 | 250 |
| 250 | 300 |

### Reaction Conditions-Hydrogen Mole Ratio

For reactions using each catalyst/support combination (each row) in Table 1 used in the hydrogenation reaction of Step (i), the mole ratio of hydrogen to 1,1,2-trichloro-1,2,2-trifluoroethane (CFC-113) may be as little about 2:1, about 3:1, about 4:1, about 5:1, about 5.5:1 or as great as about 6:1, about 6.5:1, about 7.5:1 or about 8:1, about 12:1, about 15:1, or about 20:1, for example, or within any range encompassed by two of the foregoing values as endpoints. The mole ratio of hydrogen to CFC-113 may be preferably from about 3:1 about to 15:1, and more preferably from about 4:1 to about 10:1.

### Products—Selectivity to Desired Product (HFC-143)

As demonstrated by the Examples herein, for reactions using each catalyst/support combination (each row) in Table 1, the hydrogenation Step (i) may achieve a selectivity to the 1,1,2-trifluoroethane (HFC-143) product of greater than about 20%, greater than about 30%, greater than about 40%, greater than about 50%, greater than about 60%, greater than about 70%, greater than about 80%, greater than about 90%, and for each of the foregoing, less than or equal to 100%, or within any range encompassed by two of the foregoing values as endpoints, such as from about 20% to about 90%, from about 30% to about 80%, from about 40% to about 70%, or about 50% to about 60%, based on total moles of the organic components of the composition. Specific examples of additional suitable ranges are set forth below in Table 8.

TABLE 8
Selectivity for Desired Product 1,1,2-trifluoroethane (HFC-143)
using Catalyst (Pd and Pt) on Supports (Activated Carbon,
alpha(α)-Al₂O₃, theta(θ)-Al₂O₃, delta(δ)-Al₂O₃, or
gamma(γ)-Al₂O₃ - Each Catalyst/Support Combination (Each
Row) in Table 1 - Step (i) Hydrogenation Reaction

| From (%) | To (%) |
|---|---|
| 20 | 90 |
| 20 | 80 |
| 20 | 70 |
| 20 | 60 |
| 30 | 80 |
| 30 | 70 |
| 30 | 60 |
| 40 | 80 |
| 40 | 70 |
| 40 | 60 |
| 50 | 60 |
| 60 | 70 |
| 70 | 90 |
| 70 | 80 |
| 80 | 90 |

### Selective Tailoring of the General Step (i) Hydrogenation Reaction

As discussed below, during the hydrogenation reaction of Step (i), several intermediates and/or byproducts may be formed, some of which may be considered desired intermediates and others undesired byproducts, and that the overall reaction methods and/or specific reaction conditions of step (i) may be selectively tailored, such as with (i) catalyst conditioning, and/or (ii) reactant dilution with an inert gas, to advantageously convert desired intermediates to the desired product 1,1,2-trifluoroethane (HFC-143) and/or minimize the formation of undesired byproducts. The selective tailoring of (i) catalyst conditioning and (ii) reactant dilution to the hydrogenation reaction of Step (i) may be used independently or jointly to convert desired intermediates to the desired product (HFC-143).

In particular, the Step (i) hydrogenation reaction for producing 1,1,2-trifluoroethane (HFC-143) from 1,1,2-trichloro-1,2,2-trifluoroethane (CFC-113), using each catalyst/support combination (each row) in Table 1, may produce several desired intermediates such as 1-chloro-1,1,2-trifluoroethane (HCFC-133b), 1-chloro-1,2,2-trifluoroethane (HCFC-133), and/or 1,2-dichloro-1,1,2-trifluoroethane (HCFC-123a), as well as several undesired byproducts such as 1,1,1-trofluorothane (HFC-143a), ethane (HC-170), chloroethane (HCC-160), 1,1-difluoroethane (HFC-152a), and/or HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)).

It has been discovered that the intermediates 1-chloro-1,1,2-trifluoroethane (HCFC-133b), 1-chloro-1,2,2-trifluoroethane (HCFC-133), and/or 1,2-dichloro-1,1,2-trifluoroethane (HCFC-123a) are formed in the hydrogenation reaction of Step (i) using each catalyst/support combination (each row) in Table 1, and further, that each of 1-chloro-1,1,2-trifluoroethane (HCFC-133b), 1-chloro-1,2,2-trifluoroethane (HCFC-133), and/or 1,2-dichloro-1,1,2-trifluoroethane (HCFC-123a) may themselves be converted to 1,1,2-trifluoroethane (HFC-143). In view of this finding, 1-chloro-1,1,2-trifluoroethane (HCFC-133b), 1-chloro-1,2,2-trifluoroethane (HCFC-133), and/or 1,2-dichloro-1,1,2-trifluoroethane (HCFC-123a) may be considered “desired” intermediates because each of the foregoing may be converted to the desired product 1,1,2-trifluoroethane (HFC-143) in order to increase the overall efficiency of the process.

Additionally, it has further been discovered that undesired byproducts such as 1,1,1-trofluorothane (HFC-143a), ethane (HC-170), chloroethane (HCC-160), 1,1-difluoroethane (HFC-152a), and/or HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) may also be formed in the hydrogenation reaction of Step (i) and yet are not able to be converted to form 1,1,2-trifluoroethane (HFC-143). Therefore, the present reaction methods and conditions may be tailored to avoid and/or minimize formation of such undesired byproducts.

In this connection, as discussed below and in the Examples, the present process may be tailored via one or more approaches to both manage the formation of desired intermediates and yet also minimize the formation of undesired byproducts.

Each catalyst/support combination (each row) in Table 1 may be used in the hydrogenation reaction of Step (i). The catalyst/support combination used in the hydrogenation reaction of Step (i) may preferably be palladium metal on a carbon support (Pd/C) or platinum metal on a carbon support (Pt/C). The catalyst loading may be relatively high to encourage conversion of the less reactive intermediates. For example, the Pd loading may be from about 1 wt. % to about 5 wt. % palladium metal, such as about 1 wt. %, about 2 wt. %, about 3 wt. %, about 4 wt. %, or about 5 wt. % palladium metal, based on a combined weight of the palladium metal and the carbon support. For example, the Pt loading may be from about 1 wt. % to about 5 wt. % platinum metal, such as about 1 wt. %, about 2 wt. %, about 3 wt. %, about 4 wt. %, or about 5 wt. % platinum metal, based on a combined weight of the platinum metal and the carbon support.

A summary of the preferred catalyst and support, loading, and temperatures as discussed above are summarized in Table 9 below.

TABLE 9
Summary of Preferred Catalyst (Pd, Pt) on Support
(Carbon) - Step (i) Hydrogenation Reaction

| Catalyst | Support | Loading (wt. %) | Temperature (° C.) |
|---|---|---|---|
| Pd | Activated carbon | 0.5-10 | 100-350 |
| Pd | Activated carbon | 0.5-10 | 200-300 |
| Pd | Activated carbon | 0.5-10 | 230-290 |
| Pd | Activated carbon | 0.5-10 | 250-280 |
| Pd | Activated carbon | 1-5 | 100-350 |
| Pd | Activated carbon | 1-5 | 200-300 |
| Pd | Activated carbon | 1-5 | 230-290 |
| Pd | Activated carbon | 1-5 | 250-280 |
| Pt | Activated carbon | 0.5-10 | 100-350 |
| Pt | Activated carbon | 0.5-10 | 200-300 |
| Pt | Activated carbon | 0.5-10 | 230-290 |
| Pt | Activated carbon | 0.5-10 | 250-280 |
| Pt | Activated carbon | 1-5 | 100-350 |
| Pt | Activated carbon | 1-5 | 200-300 |
| Pt | Activated carbon | 1-5 | 230-290 |
| Pt | Activated carbon | 1-5 | 250-280 |

### (i) Catalyst Conditioning

In a first approach, for each catalyst/support combination (each row) in Table 1 used in the hydrogenation reaction of Step (i), the catalyst may be conditioned to improve the efficiency and selectivity towards the desired intermediates. The catalyst conditioning may involve carrying out the hydrogenation reaction of Step (i) at a first, high temperature initially to promote selectivity to 1,1,2-trifluoroethane (HFC-143), followed by continuing the reaction at a second, reduced temperature to moderate the formation of undesired byproducts.

### (a) Catalyst Conditioning-High Temperature Phase

During an initial high temperature phase of the Step (i) hydrogenation reaction using each catalyst/support combination (each row) in Table 1, the first reaction temperature may be at least 250° C., 255° C., 260° C., 265° C., 270° C., 275° C., 280° C., 285° C., 290° C., 295° C., 300° C., 310° C., 315° C., 320° C., 325° C., 350° C., or within any range encompassed by any two of the foregoing values as endpoints, such as from about 250° C. to about 350° C., from about 255° C. to about 325° C., from about 260° C. to about 320° C., from about 265° C. to about 315° C., from about 270° C. to about 310° C., from about 275° C. to about 300° C., from about 280° C. to about 295° C., from about 285° C. to about 290° C., for example, preferably from about 250° C. to about 325° C., more preferably from about 260° C. to about 310° C., or most preferably from about 270° C. to about 300° C.

The high temperature phase of the Step (i) hydrogenation reaction using each catalyst/support combination (each row) in Table 1 may be held at this elevated temperature for a catalyst conditioning time of at least 1 hour, at least 2 hours, at least 3 hours, at least 4 hours, at least 5 hours, at least 6 hours, at least 7 hours, at least 8 hours, at least 9 hours, or at least 10 hours, or any range using any of the foregoing values as endpoints, such as from about 1 hour to about 10 hours, from about 2 hours to about 9 hours, from about 3 hours to about 8 hours, from about 4 hours to about 7 hours, or from about 5 hours to about 6 hours, for example, preferably from about 2 hour to about 9 hours, more preferably from about 3 hours to about 8 hours, or most preferably from about 4 hours to about 7 hours. The catalyst conditioning time is different from the contact time of reactants with the catalyst during the Step (i) hydrogenation reaction.

### (b) Catalyst Conditioning—Reduced Temperature Phase

Following the initial high temperature phase of the Step (i) hydrogenation reaction using each catalyst/support combination (each row) in Table 1, the reaction may proceed in the reactor at a second, reduced temperature of 200° C., 205° C., 210° C., 215° C., 220° C., 225° C., 230° C., 235° C., 240° C., 245° C., 250° C., 255° C., 260° C., 265° C., 270° C., 275° C., or within any range encompassed by any two of the foregoing values as endpoints, wherein the second temperature is lower than the first temperature. For example, the second temperature may be from about 200° C. to about 275° C., from about 205° C. to about 270° C., from about 210° C. to about 265° C., from about 215° C. to about 260° C., from about 220° C. to about 255° C., from about 225° C. to about 250° C., from about 230° C. to about 245° C., or from about 235° C. to about 240° C., for example, preferably from about 200° C. to about 275° C., more preferably from about 210° C. to about 250° C., or most preferably from about 22° C. to about 240° C.

The reduced temperature phase of the Step (i) hydrogenation reaction using each catalyst/support combination (each row) in Table 1 may be held at this reduced temperature for a catalyst runtime of at least 1 hour, at least 2 hours, at least 3 hours, at least 4 hours, at least 5 hours, at least 6 hours, at least 7 hours, at least 8 hours, at least 9 hours, at least 10 hours, at least 50 hours, at least 100 hours, at least 500 hours, at least 1000 hours, at least 2000 hours, at least 3000 hours, at least 4000 hours, at least 5000 hours, at least 6000 hours, or more, or any range using any two of the foregoing values as endpoints, such as from about 1 hour to about 6000 hours, from about 2 hours to about 5000 hours, from about 3 hours to about 4000 hours, from about 3 hours to about 3000 hours, from about 4 hours to about 2000 hours, from about 5 hours to about 1000 hours, from about 6 hours to about 500 hours, from about 7 hours to about 100 hours, or from about 8 hours to about 50 hours, or from about 9 hours to about 10 hours, for example, preferably at least 10 hours, more preferably at least 20 hours, and most preferably at least 100 hours. The catalyst runtime is the total catalyst lifetime before the catalyst will need to be regenerated, or discarded and replaced. The catalyst runtime is different from the contact time of reactants with the catalyst during the Step (i) hydrogenation reaction.

As demonstrated in the Examples, the selectivity to desired products for the Step (i) hydrogenation reaction using each catalyst/support combination (each row) in Table 1 can be further improved after an extended period of time, for example after 300 hours of operation at the reduced temperature.

The above process, in which the hydrogenation reaction of Step (i) is initially carried out at an elevated temperature, is referred to herein as catalyst conditioning. After the catalyst has been conditioned, it has been found that the Step (i) hydrogenation reaction produces a product composition in which amounts of formed desired products and intermediates are managed and yet the formation of undesired byproducts is minimized.

Products of Step (i)—Catalyst Conditioning

In particular, following catalyst conditioning, the hydrogenation reaction of Step (i) using each catalyst/support combination (each row) in Table 1, referring to FIG. 1, may produce a product composition in stream 120 from the reactor 112 comprising 1,1,2-trifluoroethane (HFC-143), as well as 1-chloro-1,1,2-trifluoroethane (HCFC-133b), 1-chloro-1,2,2-trifluoroethane (HCFC-133), and/or 1,2-dichloro-1,1,2-trifluoroethane (HCFC-123a) as desired intermediates, and potentially very minor amounts, such as less than 0.1 mol %, of unreacted 1,1,2-trichloro-1,2,2-trifluoroethane (CFC-113).

In one example, the hydrogenation reaction of Step (i) may be carried out using palladium metal catalyst on a carbon support (Pd/C) with the Pd loading from about 1 wt. % to about 5 wt. % palladium metal based on a combined weight of the palladium metal and the carbon support, wherein the catalyst may be conditioned at a temperature from about 250° C. to about 350° C. for about 1 to about 10 hours, followed by reducing the temperature to about 200° C. to about 275° C. and carrying out the reaction at the reduced temperature for at least about 1 hour to at least about 6000 hours or more.

In another example, the hydrogenation reaction of Step (i) may be carried out using palladium metal catalyst on a carbon support (Pd/C) with the Pd loading from about 3 wt. % to about 5 wt. % palladium metal based on a combined weight of the palladium metal and the carbon support, wherein the catalyst may be conditioned at a temperature from about 270° C. to about 300° C. for about 2 to about 10 hours, followed by reducing the temperature to about 200° C. to about 275° C. and carrying out the reaction at the reduced temperature for at least about 1 hour to at least about 4000 hours or more.

In another example, the hydrogenation reaction of Step (i) may be carried out using palladium metal catalyst on a carbon support (Pd/C) with the Pd loading of about 4 wt. % palladium metal based on a combined weight of the palladium metal and the carbon support, wherein the catalyst may be conditioned at a temperature of from about 275° C. to about 290° C. for about 2 to about 8 hours, followed by reducing the temperature to about 200° C. to about 275° C. and carrying out the reaction at the reduced temperature for at least about 1 hour to at least about 2000 hours or more.

In one example, the hydrogenation reaction of Step (i) may be carried out using platinum metal catalyst on a carbon support (Pt/C) with the Pt loading from about 1 wt. % to about 5 wt. % platinum metal based on a combined weight of the platinum metal and the carbon support, wherein the catalyst may be conditioned at a temperature from about 250° C. to about 350° C. for about 1 to about 10 hours, followed by reducing the temperature to about 200° C. to about 275° C. and carrying out the reaction at the reduced temperature for at least about 1 hour to at least about 6000 hours or more.

In another example, the hydrogenation reaction of Step (i) may be carried out using platinum metal catalyst on a carbon support (Pt/C) with the Pt loading from about 3 wt. % to about 5 wt. % platinum metal based on a combined weight of the platinum metal and the carbon support, wherein the catalyst may be conditioned at a temperature from about 270° C. to about 300° C. for about 2 to about 10 hours, followed by reducing the temperature to about 200° C. to about 275° C. and carrying out the reaction at the reduced temperature for at least about 1 hour to at least about 4000 hours or more.

In another example, the hydrogenation reaction of Step (i) may be carried out using platinum metal catalyst on a carbon support (Pt/C) with the Pt loading of about 4 wt. % platinum metal based on a combined weight of the platinum metal and the carbon support, wherein the catalyst may be conditioned at a temperature from about 275° C. to about 290° C. for about 2 to about 8 hours, followed by reducing the temperature to about 200° C. to about 275° C. and carrying out the reaction at the reduced temperature for at least about 1 hour to at least about 2000 hours or more.

Product Composition of Step (i)—Catalyst Conditioning-Desired Product (HFC-143)

For a hydrogenation reaction of Step (i) carried out using each catalyst/support combination (each row) in Table 1 with catalyst conditioning, referring to FIG. 1, the amount of 1,1,2-trifluoroethane (HFC-143) in the product composition in stream 120 from the reactor 112 may be at least 40 mol %, at least 50 mol %, at least 60 mol %, or at least 70 mol %, and for each of the foregoing, less than or equal to 100 mol %, or any range using any two of the foregoing values as endpoints, such as from about 40 mol % to about 70 mol %, or from about 50 mol % to about 60 mol %, based on total moles of the organic components of the composition.

Product Composition of Step (i)—Catalyst Conditioning-Desired Intermediates

For a hydrogenation reaction of Step (i) carried out using each catalyst/support combination (each row) in Table 1 with catalyst conditioning, referring to FIG. 1, the amount of 1-chloro-1,1,2-trifluoroethane (HCFC-133b) in the product composition in stream 120 from the reactor 112, if present, may be greater than 0.5 mol % and yet less than 25 mol %, less than 15 mol %, or less than 10 mol %, for example, based on total moles of the organic components of the composition.

For a hydrogenation reaction of Step (i) carried out using each catalyst/support combination (each row) in Table 1 with catalyst conditioning, referring to FIG. 1, the amount of 1-chloro-1,2,2-trifluoroethane (HCFC-133) in the product composition in stream 120 from the reactor 112, if present, may be greater than 0.5 mol % and yet less than 10 mol %, less than 5 mol %, or less than 1 mol %, for example, based on total moles of the organic components of the composition.

For a hydrogenation reaction of Step (i) carried out using each catalyst/support combination (each row) in Table 1 with catalyst conditioning, referring to FIG. 1, the amount of 1,2-dichloro-1,1,2-trifluoroethane (HCFC-123a) in the product composition in stream 120 from the reactor 112, if present, may be greater than 0.1 mol % and yet less than 10 mol %, less than 5 mol %, or less than 1 mol %, for example, based on total moles of the organic components of the composition.

For a hydrogenation reaction of Step (i) carried out using each catalyst/support combination (each row) in Table 1 with catalyst conditioning, referring to FIG. 1, the total amount of 1,1,2-trifluoroethane (HFC-143), 1-chloro-1,1,2-trifluoroethane (HCFC-133b), 1-chloro-1,2,2-trifluoroethane (HCFC-133), and 1,2-dichloro-1,1,2-trifluoroethane (HCFC-123a) in the product composition in stream 120 from the reactor 112 may be at least 80 mol %, at least 85 mol %, or at least 90 mol %, and for each of the foregoing, less than or equal to 100 mol %, for example, based on total moles of the organic components of the composition.

For example, for a hydrogenation reaction of Step (i) carried out using each catalyst/support combination (each row) in Table 1 with catalyst conditioning, referring to FIG. 1, in the product composition, the amount of 1,1,2-trifluoroethane (HFC-143) in the product composition in stream 120 from the reactor 112 may be at least 55 mol % and less than or equal to 98.9 mol %, the amount of 1-chloro-1,1,2-trifluoroethane (HCFC-133b) may be at least 0.5 mol % and less than or equal to 25 mol %, the amount of 1-chloro-1,2,2-trifluoroethane (HCFC-133) may be at least 0.5 mol % and less than or equal to 10 mol %, and the amount of 1,2-dichloro-1,1,2-trifluoroethane (HCFC-123a) may be at least 0.1 mol % and less than or equal to 10 mol %, based on the combined total moles of the HFC-143, HCFC-133b, HCFC-133, and HCFC-123a in the product composition.

The combined amount of HFC-143, HCFC-133b, HCFC-133, and HCFC-123 in the composition set forth in the preceding paragraph may be at least 85 mol % and less than or equal to 100% of the total moles of organic components of the product composition in stream 120 from the reactor 112, while the combined amount of other components including undesired byproducts (e.g., HFC-143a, HC-170, HCC-160, HFC-152, and HCFC-142 isomers) may be greater than or equal to 0 mol % and less than 15 mol % of the total moles of organic components of the product composition in stream 120 from the reactor 112.

For example, for a hydrogenation reaction of Step (i) carried out using each catalyst/support combination (each row) in Table 1 with catalyst conditioning, referring to FIG. 1, in the product composition, the amount of 1,1,2-trifluoroethane (HFC-143) in the product composition in stream 120 from the reactor 112 may be at least 75 mol % and less than or equal to 98.9 mol %, the amount of 1-chloro-1,1,2-trifluoroethane (HCFC-133b) may be at least 0.5 mol % and less than or equal to 15 mol %, the amount of 1-chloro-1,2,2-trifluoroethane (HCFC-133) may be at least 0.5 mol % and less than or equal to 5 mol %, and the amount of 1,2-dichloro-1,1,2-trifluoroethane (HCFC-123a) may be at least 0.1 mol % and less than or equal to 5 mol %, based on the combined total moles of the HFC-143, HCFC-133b, HCFC-133, and HCFC-123a in the product composition.

The combined amount of HFC-143, HCFC-133b, HCFC-133, and HCFC-123 in the composition set forth in the preceding paragraph may be at least 90 mol % and less than or equal to 100% of the total moles of organic components of the product composition in stream 120 from the reactor 112, while the combined amount of other components including undesired byproducts (e.g., HFC-143a, HC-170, HCC-160, HFC-152, and HCFC-142 isomers) may be greater than or equal to 0 mol % and less than 10 mol % of the total moles of organic components of the product composition in stream 120 from the reactor 112.

For example, for a hydrogenation reaction of Step (i) carried out using each catalyst/support combination (each row) in Table 1 with catalyst conditioning, referring to FIG. 1, in the product composition, the amount of 1,1,2-trifluoroethane (HFC-143) in the product composition in stream 120 from the reactor 112 may be at least 84 mol % and less than or equal to 98.9 mol %, the amount of 1-chloro-1,1,2-trifluoroethane (HCFC-133b) may be at least 0.5 mol % and less than or equal to 10 mol %, the amount of 1-chloro-1,2,2-trifluoroethane (HCFC-133) may be at least 0.5 mol % and less than or equal to 5 mol %, and the amount of 1,2-dichloro-1,1,2-trifluoroethane (HCFC-123a) may be at least 0.1 mol % and less than or equal to 1 mol %, based on the combined total moles of the HFC-143, HCFC-133b, HCFC-133, and HCFC-123a in the product composition.

The combined amount of HFC-143, HCFC-133b, HCFC-133, and HCFC-123 in the composition set forth in the preceding paragraph may be at least 95 mol % and less than or equal to 100% of the total moles of organic components of the product composition in stream 120 from the reactor 112, while the combined amount of other components including undesired byproducts (e.g., HFC-143a, HC-170, HCC-160, HFC-152, and HCFC-142 isomers) may be greater than or equal to 0 mol % and less than 5 mol % of the total moles of organic components of the product composition in stream 120 from the reactor 112.

Product Composition of Step (i)—Catalyst Conditioning-Undesired Byproducts

Further, following catalyst conditioning, for a hydrogenation reaction of Step (i) carried out using each catalyst/support combination (each row) in Table 1 with catalyst conditioning, referring to FIG. 1, the hydrogenation reaction may produce a product composition in stream 120 from the reactor 112 comprising 1,1,2-trifluoroethane (HFC-143) in which one or more undesired byproducts including 1,1,1-trofluorothane (HFC-143a), ethane (HC-170), chloroethane (HCC-160), 1,1-difluoroethane (HFC-152a), and/or HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) is minimized.

For a hydrogenation reaction of Step (i) carried out using each catalyst/support combination (each row) in Table 1 with catalyst conditioning, referring to FIG. 1, the amount of 1,1,1-trifluoroethane (HFC-143a) in the product composition in stream 120 from the reactor 112, if present, may be less than 2 mol %, less than 1 mol %, or less than 0.5 mol %, and for each of the foregoing, greater than or equal to 0 mol %, for example, based on total moles of the organic components of the composition.

For a hydrogenation reaction of Step (i) carried out using each catalyst/support combination (each row) in Table 1 with catalyst conditioning, referring to FIG. 1, the amount of ethane (HC-170) in the product composition in stream 120 from the reactor 112, if present, may be less than 10 mol %, less than 5 mol %, or less than 2 mol %, and for each of the foregoing, greater than or equal to 0 mol %, for example, based on total moles of the organic components of the composition.

For a hydrogenation reaction of Step (i) carried out using each catalyst/support combination (each row) in Table 1 with catalyst conditioning, referring to FIG. 1, the amount of chloroethane (HCC-160) in the product composition in stream 120 from the reactor 112, if present, may be less than 3 mol %, less than 1 mol %, or less than 0.5 mol %, and for each of the foregoing, greater than or equal to 0 mol %, for example, based on total moles of the organic components of the composition.

For a hydrogenation reaction of Step (i) carried out using each catalyst/support combination (each row) in Table 1 with catalyst conditioning, referring to FIG. 1, the amount of 1,1-difluoroethane (HFC-152a) in the product composition in stream 120 from the reactor 112, if present, may be less than 15 mol %, less than 10 mol %, or less than 5 mol %, and for each of the foregoing, greater than or equal to 0 mol %, for example, based on total moles of the organic components of the composition.

For a hydrogenation reaction of Step (i) carried out using each catalyst/support combination (each row) in Table 1 with catalyst conditioning, referring to FIG. 1, the amount of HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) in the product composition in stream 120 from the reactor 112, if present, may be less than 5 mol %, less than 3 mol % less than 2 mol %, or less than 1 mol %, and for each of the foregoing, greater than or equal to 0 mol %, for example, based on total moles of the organic components of the composition.

For a hydrogenation reaction of Step (i) carried out using each catalyst/support combination (each row) in Table 1 with catalyst conditioning, referring to FIG. 1, the total amount of 1, 1,1-trofluorothane (HFC-143a), ethane (HC-170), chloroethane (HCC-160), 1,1-difluoroethane (HFC-152a), and HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) in the product composition in stream 120 from the reactor 112, if present, may be less than 20 mol %, less than 15 mol %, or less than 10 mol %, and for each of the foregoing, greater than or equal to 0 mol %, for example, based on total moles of the organic components of the composition.

For a hydrogenation reaction of Step (i) carried out using each catalyst/support combination (each row) in Table 1 with catalyst conditioning, referring to FIG. 1, the composition in stream 120 from the reactor 112 may also include a total amount of less than 1.5 mol %, less than 1.0 mol %, or less than 0.5 mol %, for example, of all other organic components, such as R-1132a, R-1123, R-142 isomers, R-161, R-152, R-151a, and/or unknown organic components, based on total moles of the organic components of the composition.

For example, for a hydrogenation reaction of Step (i) carried out using each catalyst/support combination (each row) in Table 1 with catalyst conditioning, referring to FIG. 1, in the product composition in stream 120 from the reactor 112, the amount of 1,1,1-trifluoroethane (HFC-143a) may be less than about 2 mol % and greater than or equal to 0 mol %, the amount of ethane (HC-170) may be less than about 10 mol % and greater than or equal to 0 mol %, the amount of chloroethane (HCC-160) may be less than about 3 mol % and greater than or equal to 0 mol %, the amount of 1,1-difluoroethane (HFC-152a) may be less than about 15 mol % and greater than or equal to 0 mol %, the amount of HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) may be less than about 5 mol % and greater than or equal to 0 mol %, and the combined total amount of 1,1,1-trofluorothane (HFC-143a), ethane (HC-170), chloroethane (HCC-160), 1,1-difluoroethane (HFC-152a), and HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) in the product composition may be less than about 20 mol % and greater than or equal to 0 mol %, based on total moles of the organic components of the composition.

For example, for a hydrogenation reaction of Step (i) carried out using each catalyst/support combination (each row) in Table 1 with catalyst conditioning, referring to FIG. 1, in the product composition in stream 120 from the reactor 112, the amount of 1,1,1-trifluoroethane (HFC-143a) may be less than about 1 mol % and greater than or equal to 0 mol %, the amount of ethane (HC-170) may be less than about 5 mol % and greater than or equal to 0 mol %, the amount of chloroethane (HCC-160) may be less than about 1 mol % and greater than or equal to 0 mol %, the amount of 1,1-difluoroethane (HFC-152a) may be less than about 10 mol % and greater than or equal to 0 mol %, the amount of HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) may be less than about 3 mol % and greater than or equal to 0 mol %, and the combined total amount of 1,1,1-trofluorothane (HFC-143a), ethane (HC-170), chloroethane (HCC-160), 1,1-difluoroethane (HFC-152a), and HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) in the product composition may be less than about 15 mol % and greater than or equal to 0 mol %, based on total moles of the organic components of the composition.

For example, for a hydrogenation reaction of Step (i) carried out using each catalyst/support combination (each row) in Table 1 with catalyst conditioning, referring to FIG. 1, in the product composition in stream 120 from the reactor 112, the amount of 1,1,1-trifluoroethane (HFC-143a) may be less than about 0.5 mol % and greater than or equal to 0 mol %, the amount of ethane (HC-170) may be less than about 2 mol % and greater than or equal to 0 mol %, the amount of chloroethane (HCC-160) may be less than about 0.5 mol % and greater than or equal to 0 mol %, the amount of 1,1-difluoroethane (HFC-152a) may be less than about 5 mol % and greater than or equal to 0 mol %, the amount of HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) may be less than about 2 mol % and greater than or equal to 0 mol %, and the combined total amount of 1,1,1-trofluorothane (HFC-143a), ethane (HC-170), chloroethane (HCC-160), 1,1-difluoroethane (HFC-152a), and HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) in the product composition may be less than about 10 mol % and greater than or equal to 0 mol %, based on total moles of the organic components of the composition.

### (ii) Reactant Dilution

In a second approach to selectively tailor the reaction, using each catalyst/support combination (each row) in Table 1, the reactants used in the hydrogenation reaction of Step (i) may be diluted with an inert gas to improve selectivity to the desired product 1,1,2-trifluoroethane (HFC-143) and mitigate formation of undesired byproducts. Suitable inert gases include nitrogen, argon, for example. The second approach for selectively tailoring the hydrogenation reaction of Step (i) may be used in addition to, or as an alternative to, the catalyst conditioning.

The percentage of the volumetric flow rate of the inert gas relative to the total volumetric flow rate of the reactants in the hydrogenation reaction of Step (i) using each catalyst/support combination (each row) in Table 1 may be greater than about 0%, greater than about 1%, greater than about 5%, greater than about 10%, greater than about 20%, greater than about 50%, or greater than about 60%, or any range using any two of the foregoing values as endpoints, such as from about 0% to about 60%, from about 1% to about 50%, from about 5% to about 20%, or about 5% to about 10%. The preferred range may be from about 5% to about 50%, and the more preferred range may be from about 20% to about 40%.

Products of Step (i)—Reactant Dilution

For the hydrogenation reaction of Step (i) using each catalyst/support combination (each row) in Table 1, and further using reactant dilution, the selectivity to 1,1,2-trifluoroethane (HFC-143) may be increased by 1-2% as demonstrated in the Examples. As a result, the selectivity to 1,1,2-trifluoroethane (HFC-143) may exceed 70%, and the total selectivity to 1,1,1-trofluorothane (HFC-143a), ethane (HC-170), 1,1-difluoroethane (HFC-152a), and HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) may be less than 7%.

For a hydrogenation reaction of Step (i) carried out using each catalyst/support combination (each row) in Table 1 with reactant dilution, referring to FIG. 1, the product composition in stream 120 from the reactor 112 may comprise 1,1,2-trifluoroethane (HFC-143), as well as 1-chloro-1,1,2-trifluoroethane (HCFC-133b), 1-chloro-1,2,2-trifluoroethane (HCFC-133), and/or 1,2-dichloro-1,1,2-trifluoroethane (HCFC-123a) as desired intermediates, and potentially very minor amounts, such as less than 0.1 mol %, of unreacted 1,1,2-trichloro-1,2,2-trifluoroethane (CFC-113), based on total moles of the organic components of the composition.

### Product Composition—Desired Product (HFC-143)

For a hydrogenation reaction of Step (i) carried out using each catalyst/support combination (each row) in Table 1 with reactant dilution, referring to FIG. 1, the amount of 1,1,2-trifluoroethane (HFC-143) in the product composition in stream 120 from the reactor 112 may be at least 40 mol %, at least 50 mol %, at least 60 mol %, or at least 70 mol %, and for each of the foregoing, less than or equal to 100 mol %, or any range using any two of the foregoing values as endpoints, such as from about 40% to about 70%, or about 50% to about 60%, based on total moles of the organic components of the composition.

### Product Composition-Desired Intermediates

For a hydrogenation reaction of Step (i) carried out using each catalyst/support combination (each row) in Table 1 with reactant dilution, referring to FIG. 1, the amount of 1-chloro-1,1,2-trifluoroethane (HCFC-133b) in the product composition in stream 120 from the reactor 112 may be at least 0.5 mol % and yet less than 25 mol %, less than 15 mol %, or less than 10 mol %, for example, based on total moles of the organic components of the composition.

For a hydrogenation reaction of Step (i) carried out using each catalyst/support combination (each row) in Table 1 with reactant dilution, referring to FIG. 1, the amount of 1-chloro-1,2,2-trifluoroethane (HCFC-133) in the product composition in stream 120 from the reactor 112 may be at least 0.5 mol % and yet less than 10 mol %, less than 5 mol %, or less than 1 mol %, for example, based on total moles of the organic components of the composition.

For a hydrogenation reaction of Step (i) carried out using each catalyst/support combination (each row) in Table 1 with reactant dilution, referring to FIG. 1, the amount of 1,2-dichloro-1,1,2-trifluoroethane (HCFC-123a) in the product composition in stream 120 from the reactor 112 may be at least 0.1 mol % and yet less than 10 mol %, less than 5 mol %, or less than 1 mol %, for example, based on total moles of the organic components of the composition.

For example, for a hydrogenation reaction of Step (i) carried out using each catalyst/support combination (each row) in Table 1 with reactant dilution, referring to FIG. 1, in the product composition in stream 120 from the reactor 112, the amount of 1,1,2-trifluoroethane (HFC-143) in the product composition may be at least 55 mol % and less than or equal to 98.9 mol %, the amount of 1-chloro-1,1,2-trifluoroethane (HCFC-133b) may be at least 0.5 mol % and less than or equal to 25 mol %, the amount of 1-chloro-1,2,2-trifluoroethane (HCFC-133) may be at least 0.5 mol % and less than or equal to 10 mol %, and the amount of 1,2-dichloro-1,1,2-trifluoroethane (HCFC-123a) may be at least 0.1 mol % and less than or equal to 10 mol %, based on the combined total moles of the HFC-143, HCFC-133b, HCFC-133, and HCFC-123a in the product composition.

The combined amount of HFC-143, HCFC-133b, HCFC-133, and HCFC-123 in the composition set forth in the preceding paragraph may be at least 85 mol % and less than or equal to 100% of the total moles of organic components of the product composition in stream 120 from the reactor 112, while the combined amount of other components including undesired byproducts (e.g., HFC-143a, HC-170, HCC-160, HFC-152, and HCFC-142 isomers) may be greater than or equal to 0 mol % and less than 15 mol % of the total moles of organic components of the product composition in stream 120 from the reactor 112.

For example, for a hydrogenation reaction of Step (i) carried out using each catalyst/support combination (each row) in Table 1 with reactant dilution, referring to FIG. 1, in the product composition in stream 120 from the reactor 112, the amount of 1,1,2-trifluoroethane (HFC-143) in the product composition may be at least 75 mol % and less than or equal to 98.9 mol %, the amount of 1-chloro-1,1,2-trifluoroethane (HCFC-133b) may be at least 0.5 mol % and less than or equal to 15 mol %, the amount of 1-chloro-1,2,2-trifluoroethane (HCFC-133) may be at least 0.5 mol % and less than or equal to 5 mol %, and the amount of 1,2-dichloro-1,1,2-trifluoroethane (HCFC-123a) may be at least 0.1 mol % and less than or equal to 5 mol %, based on the combined total moles of the HFC-143, HCFC-133b, HCFC-133, and HCFC-123a in the product composition.

The combined amount of HFC-143, HCFC-133b, HCFC-133, and HCFC-123 in the composition set forth in the preceding paragraph may be at least 90 mol % and less than or equal to 100% of the total moles of organic components of the product composition in stream 120 from the reactor 112, while the combined amount of other components including undesired byproducts (e.g., HFC-143a, HC-170, HCC-160, HFC-152, and HCFC-142 isomers) may be greater than or equal to 0 mol % and less than 10 mol % of the total moles of organic components of the product composition in stream 120 from the reactor 112.

For example, for a hydrogenation reaction of Step (i) carried out using each catalyst/support combination (each row) in Table 1 with reactant dilution, referring to FIG. 1, in the product composition in stream 120 from the reactor 112, the amount of 1,1,2-trifluoroethane (HFC-143) in the product composition may be at least 84 mol % and less than or equal to 98.9 mol %, the amount of 1-chloro-1,1,2-trifluoroethane (HCFC-133b) may be at least 0.5 mol % and less than or equal to 10 mol %, the amount of 1-chloro-1,2,2-trifluoroethane (HCFC-133) may be at least 0.5 mol % and less than or equal to 5 mol %, and the amount of 1,2-dichloro-1,1,2-trifluoroethane (HCFC-123a) may be at least 0.1 mol % and less than or equal to 1 mol %, based on the combined total moles of the HFC-143, HCFC-133b, HCFC-133, and HCFC-123a in the product composition.

The combined amount of HFC-143, HCFC-133b, HCFC-133, and HCFC-123 in the composition set forth in the preceding paragraph may be at least 95 mol % and less than or equal to 100% of the total moles of organic components of the product composition in stream 120 from the reactor 112, while the combined amount of other components including undesired byproducts (e.g., HFC-143a, HC-170, HCC-160, HFC-152, and HCFC-142 isomers) may be greater than or equal to 0 mol % and less than 5 mol % of the total moles of organic components of the product composition in stream 120 from the reactor 112.

For a hydrogenation reaction of Step (i) carried out using each catalyst/support combination (each row) in Table 1 with reactant dilution, referring to FIG. 1, the total amount of 1,1,2-trifluoroethane (HFC-143), 1-chloro-1,1,2-trifluoroethane (HCFC-133b), 1-chloro-1,2,2-trifluoroethane (HCFC-133), and 1,2-dichloro-1,1,2-trifluoroethane (HCFC-123a) in the product composition in stream 120 from the reactor 112 may be at least 80 mol %, at least 85 mol %, or at least 90 mol %, and for each of the foregoing, less than or equal to 100 mol %, for example, based on total moles of the organic components of the composition.

### Product Composition—Undesired Byproducts

Further, for a hydrogenation reaction of Step (i) carried out using each catalyst/support combination (each row) in Table 1 with reactant dilution, referring to FIG. 1, the hydrogenation reaction may produce a product composition in stream 120 from the reactor 112 comprising 1,1,2-trifluoroethane (HFC-143) in which one or more undesired byproducts including 1,1,1-trofluorothane (HFC-143a), ethane (HC-170), chloroethane (HCC-160), 1,1-difluoroethane (HFC-152a), and/or HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) is minimized.

For a hydrogenation reaction of Step (i) carried out using each catalyst/support combination (each row) in Table 1 with reactant dilution, referring to FIG. 1, the amount of 1,1,1-trifluoroethane (HFC-143a) in the product composition in stream 120 from the reactor 112 may be less than 2 mol %, less than 1 mol %, or less than 0.5 mol %, and for each of the foregoing, greater than or equal to 0 mol %, for example, based on total moles of the organic components of the composition.

For a hydrogenation reaction of Step (i) carried out using each catalyst/support combination (each row) in Table 1 with reactant dilution, referring to FIG. 1, the amount of ethane (HC-170) in the product composition in stream 120 from the reactor 112 may be less than 10 mol %, less than 5 mol %, or less than 2 mol %, and for each of the foregoing, greater than or equal to 0 mol %, for example, based on total moles of the organic components of the composition.

For a hydrogenation reaction of Step (i) carried out using each catalyst/support combination (each row) in Table 1 with reactant dilution, referring to FIG. 1, the amount of chloroethane (HCC-160) in the product composition in stream 120 from the reactor 112 may be less than 3 mol %, less than 1 mol %, or less than 0.5 mol %, and for each of the foregoing, greater than or equal to 0 mol %, for example, based on total moles of the organic components of the composition.

For a hydrogenation reaction of Step (i) carried out using each catalyst/support combination (each row) in Table 1 with reactant dilution, referring to FIG. 1, the amount of 1,1-difluoroethane (HFC-152a) in the product composition in stream 120 from the reactor 112 may be less than 15 mol %, less than 10 mol %, or less than 5 mol %, and for each of the foregoing, greater than or equal to 0 mol %, for example, based on total moles of the organic components of the composition.

For a hydrogenation reaction of Step (i) carried out using each catalyst/support combination (each row) in Table 1 with reactant dilution, referring to FIG. 1, the amount of HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) in the product composition in stream 120 from the reactor 112 may be less than 5 mol %, less than 3 mol % less than 2 mol %, or less than 1 mol %, and for each of the foregoing, greater than or equal to 0 mol %, for example, based on total moles of the organic components of the composition.

For a hydrogenation reaction of Step (i) carried out using each catalyst/support combination (each row) in Table 1 with reactant dilution, referring to FIG. 1, the total amount of 1,1,1-trofluorothane (HFC-143a), ethane (HC-170), chloroethane (HCC-160), 1,1-difluoroethane (HFC-152a), and HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) in the product composition in stream 120 from the reactor 112 may be less than 20 mol %, less than 15 mol %, or less than 10 mol %, and for each of the foregoing, greater than or equal to 0 mol %, for example, based on total moles of the organic components of the composition.

For a hydrogenation reaction of Step (i) carried out using each catalyst/support combination (each row) in Table 1 with reactant dilution, referring to FIG. 1, the composition in stream 120 from the reactor 112 may also include a total amount of less than 1.5 mol %, less than 1.0 mol %, or less than 0.5 mol %, and for each of the foregoing, greater than or equal to 0 mol %, for example, of all other organic components, such as R-1132a, R-1123, R-142 isomers, R-161, R-152, R-151a, and/or unknown organic components, based on total moles of the organic components of the composition.

For example, for a hydrogenation reaction of Step (i) carried out using each catalyst/support combination (each row) in Table 1 with reactant dilution, referring to FIG. 1, in the product composition in stream 120 from the reactor 112, the amount of 1,1,1-trifluoroethane (HFC-143a) may be less than about 2 mol % and greater than or equal to 0 mol %, the amount of ethane (HC-170) may be less than about 10 mol % and greater than or equal to 0 mol %, the amount of chloroethane (HCC-160) may be less than about 3 mol % and greater than or equal to 0 mol %, the amount of 1,1-difluoroethane (HFC-152a) may be less than about 15 mol % and greater than or equal to 0 mol %, the amount of HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) may be less than about 5 mol % and greater than or equal to 0 mol %, the combined total amount of 1,1,1-trofluorothane (HFC-143a), ethane (HC-170), chloroethane (HCC-160), 1,1-difluoroethane (HFC-152a), and HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) in the product composition may be less than about 20 mol % and greater than or equal to 0 mol %, based on total moles of the organic components of the composition, and the product composition may include a total amount of less than 1.5 mol % and greater than or equal to 0 mol % of all other organic components such as R-1132a, R-1123, R-142 isomers, R-161, R-152, R-151a, and/or unknown organic components, based on total moles of the organic components of the composition.

For example, for a hydrogenation reaction of Step (i) carried out using each catalyst/support combination (each row) in Table 1 with reactant dilution, referring to FIG. 1, in the product composition in stream 120 from the reactor 112, the amount of 1,1,1-trifluoroethane (HFC-143a) may be less than about 1 mol % and greater than or equal to 0 mol %, the amount of ethane (HC-170) may be less than about 5 mol % and greater than or equal to 0 mol %, the amount of chloroethane (HCC-160) may be less than about 1 mol % and greater than or equal to 0 mol %, the amount of 1,1-difluoroethane (HFC-152a) may be less than about 10 mol % and greater than or equal to 0 mol %, the amount of HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) may be less than about 3 mol % and greater than or equal to 0 mol %, the combined total amount of 1,1,1-trofluorothane (HFC-143a), ethane (HC-170), chloroethane (HCC-160), 1,1-difluoroethane (HFC-152a), and HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) in the product composition may be less than about 15 mol % and greater than or equal to 0 mol %, based on total moles of the organic components of the composition and the product composition may include a total amount of less than 1 mol % and greater than or equal to 0 mol % of all other organic components such as R-1132a, R-1123, R-142 isomers, R-161, R-152, R-151a, and/or unknown organic components, based on total moles of the organic components of the composition.

For example, for a hydrogenation reaction of Step (i) carried out using each catalyst/support combination (each row) in Table 1 with reactant dilution, referring to FIG. 1, in the product composition in stream 120 from the reactor 112, the amount of 1, 1,1-trifluoroethane (HFC-143a) may be less than about 0.5 mol % and greater than or equal to 0 mol %, the amount of ethane (HC-170) may be less than about 2 mol % and greater than or equal to 0 mol %, the amount of chloroethane (HCC-160) may be less than about 0.5 mol % and greater than or equal to 0 mol %, the amount of 1,1-difluoroethane (HFC-152a) may be less than about 5 mol % and greater than or equal to 0 mol %, the amount of HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) may be less than about 2 mol % and greater than or equal to 0 mol %, the combined total amount of 1,1,1-trofluorothane (HFC-143a), ethane (HC-170), chloroethane (HCC-160), 1,1-difluoroethane (HFC-152a), and HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) in the product composition may be less than about 10 mol % and greater than or equal to 0 mol %, based on total moles of the organic components of the composition, and the product composition may include a total amount of less than 0.5 mol % and greater than or equal to 0 mol % of all other organic components such as R-1132a, R-1123, R-142 isomers, R-161, R-152, R-151a, and/or unknown organic components, based on total moles of the organic components of the composition.

### IV. Step (ii)

### General Process

The dehydrofluorination reaction of Step (ii) may be carried out in the vapor phase in a suitable reactor, for example a tubular reactor made from a material which is resistant to temperature and/or corrosion such as nickel and its alloys, including Hastelloy (for example, Hastelloy C276), Inconel (for example Inconel 600), Incoloy, and Monel wherein the vessels which may be lined with fluoropolymers.

The reactor may be first cleaned and flushed with an inert gas such as nitrogen, followed by packing with a catalyst such as those described below. The catalyst may be pretreated within the reactor such as by drying in the manner described further below, followed by metering the reactants into the rector to initiate the reaction.

The process flow may be in the down or up direction through a bed of the catalyst. Reactants may be flowed through a scrubber to remove by-products from the reaction, such as hydrogen fluoride (HF) and/or hydrogen chloride (HCl), and the reaction products may be collected by capture in a cooled cylinder, for example.

### Catalysts

The catalyst and process conditions play an important role in the dehydrofluorination reaction.

Suitable catalysts for the dehydrofluorination reaction include metal oxides such as chromium oxide (Cr₂O₃), aluminum oxide (Al₂O₃), iron oxide (Fe₂O₃), and magnesium oxide (MgO). Fluorination treatment of the catalyst may be conducted using anhydrous hydrogen fluoride (HF) under conditions effective to convert a portion of metal oxides into corresponding metal fluorides, such as via the procedure disclosed in U.S. Pat. No. 6,780,815 to Cerri et al., the disclosure of which is expressly incorporated by reference herein. Other suitable catalysts for the dehydrofluorination reaction include metal fluorides such as chromium fluoride (CrF₃), alumina fluoride (AlF₃), iron fluoride (FeF₃), magnesium fluoride (MgF₂), and various combinations of thereof.

Other metals, such as Pd, Pt, and Ni, may also be loaded onto the above fluorinated metal oxides, for example, via a wet impregnation process wherein a salt of the metal is exposed to the fluorinated metal oxide support in solution, followed by drying and then reduction with hydrogen gas. The metal catalysts supported on fluorinated metal oxide (resulting in a metal fluoride such as CrF₃, AlF₃, FeF₃, or MgF₂) supports are listed in Table 10 below.

TABLE 10
Supported Metal Catalysts - Step (ii) Dehydrofluorination Reaction

| Catalyst | Support |
|---|---|
| Pd | CrF₃ |
| Pd | AlF₃ |
| Pd | FeF₃ |
| Pd | MgF₂ |
| Pt | CrF₃ |
| Pt | AlF₃ |
| Pt | FeF₃ |
| Pt | MgF₂ |
| Ni | CrF₃ |
| Ni | AlF₃ |
| Ni | FeF₃ |
| Ni | MgF₂ |

Catalyst Loading (Pd, Pt, and Ni on CrF₃, AlF₃, FeF₃, or MgF₂)

The amount of metal loading on the support may be from about 0.01 wt. %, about 0.05 wt. %, about 0.1 wt. %, about 0.2 wt. %, about 0.3 wt. %, about 0.4 wt. %, about 0.5 wt. %, or about 1 wt. % to about 2 wt. %, about 3 wt. %, 5 wt. % 10 wt. %, or 20 wt. %, or 30 wt. %, or 40 wt. %, or 50 wt. % or within any range encompassed by two of the foregoing values as endpoints, such as from about 0.01 wt. % to about 50 wt. %, from about 0.05 wt. % to about 40 wt. %, from about 0.1 wt. % to about 30 wt. %, from about 0.2 wt. % to about 20 wt. %, from about 0.3 wt. % to about 10 wt. %, from about 0.4 wt. % to about 5 wt. %, from about 0.5 wt. % to about 3 wt. %, or from about 1 wt. % to about 2 wt. %, based on a total weight of the catalyst and support. For supported noble metal catalysts such as platinum or palladium, the metal loading may be ranged from about 0.01 wt. % to about 5 wt. %, preferably from about 0.05 wt. % to about 2 wt. %, and more preferably from about 0.1 wt. % to about 1 wt. %. Specific examples of additional suitable ranges are set forth below in Table 11.

TABLE 11
Loading for Catalysts (Pd, Pt, and Ni Loaded onto CrF₃,
AlF₃, FeF₃, or MgF₂) - Step (ii) Dehydrofluorination Reaction

| From (wt. %) | To (wt. %) |
|---|---|
| 0.01 | 10 |
| 0.01 | 5 |
| 0.01 | 3 |
| 0.01 | 2 |
| 0.01 | 1 |
| 0.05 | 10 |
| 0.05 | 5 |
| 0.05 | 3 |
| 0.05 | 2 |
| 0.05 | 1 |
| 0.1 | 10 |
| 0.1 | 5 |
| 0.1 | 3 |
| 0.1 | 2 |
| 0.1 | 1 |

When fluorinated alumina is used, the amount of metal loading on the support may be from about 0.01 wt. % to about 5 wt. %, preferably from about 0.05 wt. % to about 2 wt. %, most preferably from about 0.1 wt. % to about 1 wt. %.

### Catalyst BET Surface Area

The catalyst used in step (ii) may have a proper BET (Brunauer, Emmet, and Teller) surface area. The BET surface area of the catalyst may be as low as about 10 m²/g, about 20 m²/g, about 30 m²/g, about 40 m²/g, about 50 m²/g, about 60 m²/g, about 70 m²/g, about 80 m²/g, about 90 m²/g, about 100 m²/g, or as high as about 110 m²/g, about 120 m²/g, about 130 m²/g, about 140 m²/g, about 150 m²/g, about 175 m²/g, about 200 m²/g, about 225 m²/g, about 250 m²/g, about 300 m²/g, or within any range encompassed by any of the foregoing values as endpoints, such as from about 10 m²/g to about 300 m²/g, from about 20 m²/g to about 250 m²/g, from about 30 m²/g to about 225 m²/g, from about 40 m²/g to about 200 m²/g, from about 50 m²/g to about 175 m²/g, from about 60 m²/g to about 150 m²/g, from about 70 m²/g to about 140 m²/g, from about 80 m²/g to about 130 m²/g, from about 90 m²/g, to about 120 m²/g, or from about 100 m²/g to about 110 m²/g. For metal oxides catalysts, the BET surface area may be preferably greater than about 100 m²/g. For fluorinated metal oxides catalysts, the BET surface area may be preferably greater than about 20 m²/g. The BET analysis is the standard method for determining surface areas from nitrogen adsorption isotherms. The BET surface areas of catalysts may be measured using TriStar II Micromeritics instrument. Catalyst samples are degassed before the analysis using FlowPrep 060 instrument. Specific examples of additional suitable ranges are set forth below in Table 12.

TABLE 12
BET Surface Area of Catalysts (Cr₂O₃, Al₂O₃, Fe₂O₃,
MgO, CrF₃, AlF₃, FeF₃, MgF₂ or Pd, Pt, and Ni Loaded
onto CrF₃, AlF₃, FeF₃, or MgF₂) - Step (ii)
Dehydrofluorination Reaction

| From (m²/g) | To (m²/g) |
|---|---|
| 10 | 300 |
| 10 | 250 |
| 10 | 200 |
| 10 | 150 |
| 10 | 100 |
| 20 | 300 |
| 20 | 250 |
| 20 | 200 |
| 20 | 150 |
| 20 | 100 |
| 25 | 300 |
| 25 | 250 |
| 25 | 200 |
| 25 | 150 |
| 25 | 100 |

When fluorinated alumina is used, the BET surface area may be greater than about 10 m²/g, preferably greater than 20 m²/g, most preferably greater than 25 m²/g.

### Catalyst Pretreatment

The catalyst may be pretreated by drying at elevated temperatures, as low as about 200° C., about 250° C., about 300° C., about 350° C., about 360° C., about 370° C., or as high as about 390° C., about 400° C., about 450° C., about 500° C., about 550° C., about 600° C., or within any range encompassed by two of the foregoing values as endpoints, such as from about 200° C. to about 600° C., from about 250° C. to about 550° C., from about 300° C. to about 500° C., from about 350° C. to about 450° C., from about 360° C. to about 400° C., or from about 370° C. to about 390° C. Specific examples of additional suitable ranges are set forth in Table 13 below.

TABLE 13
Pre-Treatment Drying Temperatures for Catalysts
(Cr₂O₃, Al₂O₃, Fe₂O₃, MgO, CrF₃, AlF₃, FeF₃, MgF₂ or
Pd, Pt, and Ni Loaded onto CrF₃, AlF₃, FeF₃, or
MgF₂) - Step (ii) Dehydrofluorination Reaction

| From (° C.) | To (° C.) |
|---|---|
| 200 | 600 |
| 200 | 550 |
| 200 | 500 |
| 200 | 450 |
| 200 | 400 |
| 300 | 600 |
| 300 | 550 |
| 300 | 500 |
| 300 | 450 |
| 300 | 400 |
| 400 | 600 |
| 400 | 550 |
| 400 | 500 |
| 400 | 450 |

When fluorinated alumina is used, the catalyst may be pretreated by drying a temperature of from about 200° C. to about 600° C., preferably from about 300° C. to about 600° C., most preferably from about 400° C. to about 550° C.

As part of the catalyst activation, the catalyst may be exposed to an inert gas such as N₂. The pretreatment process may take as low as about 1 hour, about 2 hours, about 3 hours, about 4 hours, or as high about 5 hours, about 6 hours, about 10 hours, about 20 hours, or within any range encompassed by two of the foregoing values as endpoints such as from about 1 hour to about 20 hours, from about 2 hours to about 10 hours, from about 3 hours to about 6 hours, or from about 4 hours to about 5 hours, for example.

When fluorinated alumina is used, the pretreatment process may take from about 1 hour to about 10 hours, preferably from about 2 hours to about 6 hours, most preferably from about 3 hours to about 5 hours.

### Reaction Conditions—Temperature

The temperature range for dehydrofluorination reaction may be as low as about 125° C., about 150° C., about 200° C., about 250° C., about 300° C., about 350° C., about 400° C., about 450° C., or as high as about 500° C., about 550° C., about 600° C., about 650° C., about 700° C., about 750° C., about 800° C. or within any range encompassed by two of the foregoing values as endpoints, such as from about 125° C. to about 800° C., from about 150° C. to about 750° C., from about 200° C. to about 650° C., from about 250° C. to about 600° C., from about 300° C. to about 550° C., about 350° C. to about 500° C., or about 400° C. to about 450° C. The temperature may be preferably from about 250° C. to about 450° C., and more preferably from about 300° C. to about 400° C. Specific examples of additional suitable ranges are set forth below in Table 14.

TABLE 14
Reaction Temperature when using Catalysts (Cr₂O₃, Al₂O₃, Fe₂O₃,
MgO, CrF₃, AlF₃, FeF₃, MgF₂ or Pd, Pt, and Ni Loaded
onto CrF₃, AlF₃, FeF₃, or MgF₂) - Step (ii)
Dehydrofluorination Reaction

| From (° C.) | To (° C.) |
|---|---|
| 125 | 500 |
| 125 | 450 |
| 125 | 400 |
| 125 | 350 |
| 125 | 300 |
| 250 | 500 |
| 250 | 450 |
| 250 | 400 |
| 250 | 350 |
| 250 | 300 |
| 300 | 500 |
| 300 | 450 |
| 300 | 400 |
| 300 | 350 |

When fluorinated alumina is used, the reaction temperature may be from about 125° C. to about 500° C., preferably from about 250° C. to about 450° C., most preferably from about 300° C. to about 400° C.

### Reaction Conditions—Pressure

The pressure may be as little as about 1 psig, about 2 psig, about 3 psig, about 4 psig or about 5 psig, about 10 psig, about 15 psig, about 20 psig, about 25 psig, about 30 psig, about 35 psig, about 40 psig, about 50 psig, or within any range encompassed by two of the foregoing values as endpoints such as from about 1 psig to about 50 psig, from about 2 psig to about 40 psig, from about 3 psig to about 35 psig, from about 4 psig to about 25 psig, from about 5 psig to about 20 psig, or from about 10 psig to about 15 psig. For example, the pressure may be from about 1 psig to about 50 psig, preferably from about 5 psig to about 30 psig, and more preferably from about 10 psig to about 20 psig. Specific examples of additional suitable ranges are set forth below in Table 15.

TABLE 15
Reaction Pressure when using Catalysts (Cr₂O₃, Al₂O₃, Fe₂O₃,
MgO, CrF₃, AlF₃, FeF₃, MgF₂ or Pd, Pt, and Ni Loaded
onto CrF₃, AlF₃, FeF₃, or MgF₂) - Step (ii)
Dehydrofluorination Reaction

| From (psig) | To (psig) |
|---|---|
| 1 | 50 |
| 1 | 40 |
| 1 | 35 |
| 1 | 30 |
| 1 | 20 |
| 5 | 50 |
| 5 | 40 |
| 5 | 35 |
| 5 | 30 |
| 5 | 20 |
| 10 | 50 |
| 10 | 40 |
| 10 | 35 |
| 10 | 30 |
| 10 | 20 |

When fluorinated alumina is used, the reaction pressure may be from about 1 psig to about 50 psig, preferably from about 5 psig to about 30 psig, most preferably from about 10 psig to about 20 psig.

### Reaction Conditions—Contact Time

The contact time of the reactants with the catalyst may be as little as about 0.1 second, about 1 second, about 5 seconds, about 10 seconds, about 15 seconds or about 20 seconds, or as long as about 25 seconds, about 30 seconds, about 40 seconds, about 50 seconds, about 60 seconds, about 120 seconds, about or within any range encompassed by two of the foregoing values as endpoints, such as from about 0.1 seconds to about 120 seconds, from about 1 second to about 60 seconds, from about 5 seconds to about 50 seconds, from about 10 seconds to about 40 seconds, from about 15 seconds to about 30 seconds, or from about 20 seconds to about 25 seconds. For example, the contact time may be from about 1 second to about 60 seconds. Specific examples of additional suitable ranges are set forth below in Table 16.

TABLE 16
Contact Time of Reactants with Catalysts (Cr₂O₃, Al₂O₃, Fe₂O₃,
MgO, CrF₃, AlF₃, FeF₃, MgF₂ or Pd, Pt, and Ni Loaded
onto CrF₃, AlF₃, FeF₃, or MgF₂) - Step (ii)
Dehydrofluorination Reaction

| From (seconds) | To (seconds) |
|---|---|
| 1 | 120 |
| 1 | 60 |
| 1 | 40 |
| 1 | 30 |
| 1 | 20 |
| 5 | 120 |
| 5 | 60 |
| 5 | 40 |
| 5 | 30 |
| 5 | 20 |
| 10 | 120 |
| 10 | 60 |
| 10 | 40 |
| 10 | 30 |
| 10 | 20 |

When fluorinated alumina is used, the contact time may be from about 1 second to about 60 seconds, preferably from about 5 seconds to about 40 seconds, most preferably from about 10 seconds to about 30 seconds.

### Products—Cis/Trans Molar Ratio

In the dehydrofluorination reactions of Step (ii), the cis/trans molar ratio of the 1,2-difluoroethylene in the product mixture may be as low as about 1, about 2, about 3, about 4, about 5, about 6, about 7, or as high as about 9, about 10, about 11, about 12, about 13, about 14, about 15 or within any range encompassed by two of the foregoing values as endpoints, such as from about 1 to about 15, from about 2 to about 14, from about 3 to about 13, from about 3 to about 12, from about 5 to about 11, from about 6 to about 10, from about 7 to about 9. For example, the cis/trans ratio may be from about 2 to about 15.

When fluorinated alumina is used, the cis/trans molar ration of the 1,2-difluoroethylene in the product mixture may be from about 1 to about 15, preferably from about 1 to about 10, most preferably from about 2 to about 7.

### Products—Selectivity

The selectivity to the desired 1,2-difluoroethylene product (the sum of 1232E and 1232Z) may be as low as about 80%, about 85%, about 89% about 90%, about 91%, about 92%, or as high as about 94%, about 95% about 96%, about 97%, about 98%, about 99%, or within any range encompassed by two of the foregoing values as endpoints, such as from about 80% to about 99%, from about 85% to about 98%, from about 89% to about 97%, from about 90% to about 96%, from about 91% to about 95%, or from about 92% to about 94%. Specific examples of additional suitable ranges are set forth below in Table 17.

TABLE 17
Selectivity to Desired Product using Catalysts (Cr₂O₃, Al₂O₃, Fe₂O₃,
MgO, CrF₃, AlF₃, FeF₃, MgF₂ or Pd, Pt, and Ni
Loaded onto CrF₃, AlF₃, FeF₃, or MgF₂) - Step (ii)
Dehydrofluorination Reaction

| From (%) | To (%) |
|---|---|
| 85 | 99 |
| 85 | 98 |
| 85 | 97 |
| 85 | 96 |
| 85 | 95 |
| 90 | 99 |
| 90 | 98 |
| 90 | 97 |
| 90 | 96 |
| 90 | 95 |
| 95 | 99 |
| 95 | 98 |
| 95 | 97 |
| 95 | 96 |

When fluorinated alumina is used, the selectivity to the desired 1,2-difluoroethylene product (the sum of 1232E and 1232Z) may be from about 85% to about 99%, preferably from about 90% to about 99%, most preferably from about 95% to about 99%.

### Products—Conversion of the Starting Material

The conversion of the starting material to 1,2-difluoroethylene may be greater than about 10%, greater than about 20%, greater than about 30%, greater than about 40%, greater than about 50%, greater than about 60%, greater than about 70%, greater than about 80%, greater than about 90%, greater than about 95%, greater than about 99%, or within any range encompassed by two of the foregoing values as endpoints, such as from about 10% to about 99%, from about 20% to about 95%, from about 30% to about 80%, from about 40% to about 70%, or from about 50% to about 60%. Specific examples of additional suitable ranges are set forth below in Table 18.

TABLE 18
Conversion of Starting Material to 1,2-difluoroethylene
Catalysts (Cr₂O₃, Al₂O₃, Fe₂O₃, MgO, CrF₃, AlF₃, FeF₃,
MgF₂ or Pd, Pt, and Ni Loaded onto CrF₃, AlF₃, FeF₃, or
MgF₂) - Step (ii) Dehydrofluorination Reaction

| From (%) | To (%) |
|---|---|
| 20 | 99 |
| 20 | 95 |
| 20 | 90 |
| 20 | 80 |
| 20 | 70 |
| 30 | 99 |
| 30 | 95 |
| 30 | 90 |
| 30 | 80 |
| 30 | 70 |
| 60 | 99 |
| 60 | 95 |
| 60 | 90 |
| 60 | 80 |
| 60 | 70 |

When fluorinated alumina is used, the conversion of the starting material to 1,2-difluoroethylene may be greater than about 20%, preferably greater than about 30%, most preferably greater than about 60%.

### Catalyst Regeneration

It may also be advantageous to periodically regenerate the catalyst after prolonged use while in place in the reactor. Regeneration of the catalyst may be accomplished by any means known in the art, for example, by passing air or air diluted with nitrogen over the catalyst at temperatures of from about 100° C. to about 400° C., preferably from about 200° C. to about 375° C., for from about 0.5 hour to about 3 days. This may be followed by hydrogen fluoride treatment at temperatures of from about 100° C. to about 400° C., preferably from about 200° C. to about 350° C., for fluorinated catalysts or hydrogen treatment at temperatures of from about 100° C. to about 400° C., preferably from about 200° C. to about 350° C. for supported transition metal catalysts.

When fluorinated alumina is used, the catalyst may be regenerated by passing air or air diluted with nitrogen over the catalyst at temperatures of from about 100° C. to about 400° C., preferably from about 200° C. to about 375° C., for from about 0.5 hour to about 3 days. This may be followed by hydrogen fluoride treatment at temperatures of from about 100° C. to about 400° C., preferably from about 200° C. to about 350° C. Additionally, the present process advantageously avoids and/or minimizes formation of 1,1,1-trifluoroethane (HFC-143a) wherein the products of Step (ii), including trans-1,2-difluoroethylene (HFO-1132E), may include less than 5 wt. %, less than 3 wt. %, less than 1 wt. %, less than 0.5 wt. %, or less than 0.1 wt. % of 1,1,1-trifluoroethane (HFC-143a), based on a total weight of the product composition.

### Properties When the Catalyst is Fluorinates Alumina

When fluorinated alumina is used as a catalyst, the following properties may be present. The amount of metal loading on the support may be from about 0.01 wt. % to about 5 wt. %, preferably from about 0.05 wt. % to about 2 wt. %, most preferably from about 0.1 wt. % to about 1 wt. %. The BET surface area may be greater than about 10 m²/g, preferably greater than 20 m²/g, most preferably greater than 25 m²/g. The catalyst may be pretreated by drying a temperature of from about 200° C. to about 600° C., preferably from about 300° C. to about 600° C., most preferably from about 400° C. to about 550° C. The pretreatment process may take from about 1 hour to about 10 hours, preferably from about 2 hours to about 6 hours, most preferably from about 3 hours to about 5 hours. The reaction temperature may be from about 125° C. to about 500° C., preferably from about 250° C. to about 450° C., most preferably from about 300° C. to about 400° C. The reaction pressure may be from about 1 psig to about 50 psig, preferably from about 5 psig to about 30 psig, most preferably from about 10 psig to about 20 psig. The contact time may be from about 1 second to about 60 seconds, preferably from about 5 seconds to about 40 seconds, most preferably from about 10 seconds to about 30 seconds. The cis/trans molar ration of the 1,2-difluoroethylene in the product mixture may be from about 1 to about 15, preferably from about 1 to about 10, most preferably from about 2 to about 7. The selectivity to the desired 1,2-difluoroethylene product (the sum of 1232E and 1232Z) may be from about 85% to about 99%, preferably from about 90% to about 99%, most preferably from about 95% to about 99%. The conversion of the starting material to 1,2-difluoroethylene may be greater than about 20%, preferably greater than about 30%, most preferably greater than about 60%. The catalyst may be regenerated by passing air or air diluted with nitrogen over the catalyst at temperatures of from about 100° C. to about 400° C., preferably from about 200° C. to about 375° C., for from about 0.5 hour to about 3 days. This regeneration may be followed by hydrogen fluoride treatment at temperatures of from about 100° C. to about 400° C., preferably from about 200° C. to about 350° C.

### V. Step (iii)

### General Process

The 1,2-difluoroethylene (HFO-1132) obtained in step (ii) above may be produced as a mixture containing both the trans-1,2-difluoroethylene (HFO-1132E) and cis-1,2-difluoroethylene (HFO-1132Z) isomers.

In step (iii), the cis-1,2-difluoroethylene (HFO-1132Z) isomer may be converted to the trans-1,2-difluoroethylene (HFO-1132E) isomer either by exposure to heat and/or a catalyst to yield a final product comprising at least 5 wt. % trans-1,2-difluoroethylene (HFO-1232E), at least 10 wt. % trans-1,2-difluoroethylene (HFO-1232E), at least 20 wt. % trans-1,2-difluoroethylene (HFO-1232E), at least 30 wt. % trans-1,2-difluoroethylene (HFO-1232E), or greater. The product stream from isomerization reactor can be further distilled to yield a final product comprising, consisting essentially of, or consisting of, the trans-1,2-difluoroethylene (HFO-1132E) isomer in high purity, such as at least about 95 wt. %, at least about 99.0 wt. %, at least about 99.9 wt. %, at least about 99.99 wt. % or greater.

The isomerization reaction may be conducted in any suitable reaction vessel or reactor, but it should preferably be constructed from materials which are resistant to corrosion such as nickel and its alloys, including Hastelloy (for example, Hastelloy C276), Inconel (for example Inconel 600), and Monel wherein the vessels which may be lined with fluoropolymers. These may be single pipe or multiple tubes packed with an isomerization catalyst.

### Reaction Conditions—Temperature

The temperature range for the isomerization reaction may be as low as about 100° C., about 150° C., about 200° C., about 250° C., about 300° C., about 350° C., about 400° C., about 450° C., or as high as about 500° C., about 550° C., about 600° C., about 650° C., about 700° C., about 750° C., about 800° C. or within any range encompassed by two of the foregoing values as endpoints, such as from about 100° C. to about 800° C., from about 150° C. to about 750° C., from about 200° C. to about 700° C., from about 250° C. to about 650° C., from about 300° C. to about 600° C., from about 350° C. to about 550° C., or from about 400° C. to about 500° C. Specific examples of additional suitable ranges are set forth below in Table 19.

TABLE 19
Reaction Temperature - Step (iii) Isomerization Reaction

| From (° C.) | To (° C.) |
|---|---|
| 250 | 800 |
| 250 | 750 |
| 250 | 700 |
| 250 | 650 |
| 250 | 600 |
| 250 | 500 |
| 300 | 800 |
| 300 | 750 |
| 300 | 700 |
| 300 | 650 |
| 300 | 600 |
| 300 | 500 |

### Reaction Conditions—Pressure

The reaction may be conducted at atmospheric pressure, super-atmospheric pressure or under vacuum. The vacuum pressure can be from about 5 torr, about 10 torr, about 25 torr, about 50 torr, about 100 torr, about 150 torr, about 200 torr, about 250 torr, about 300 torr, to about 350 torr, 400 torr, about 450 torr, about 500 torr, about 550 torr, about 600 torr, about 650 torr, about 700 torr, about 760 torr, or any range using any of the foregoing values as endpoints, such as from about 5 torr to about 760 torr, about 10 torr to about 700 torr, about 25 torr to about 650 torr, about 50 torr to about 600 torr, about 100 torr to about 550 torr, about 150 torr to about 500 torr, about 200 torr about 450 torr, or about 250 torr to about 400 torr, or about 300 torr to about 350 torr. Specific examples of additional suitable ranges are set forth below in Table 20.

TABLE 20
Reaction Pressure - Step (iii) Isomerization Reaction

| From (torr) | To (torr) |
|---|---|
| 5 | 760 |
| 5 | 600 |
| 5 | 500 |
| 5 | 400 |
| 5 | 300 |
| 5 | 200 |
| 50 | 760 |
| 50 | 600 |
| 50 | 500 |
| 50 | 400 |
| 50 | 300 |
| 50 | 200 |
| 100 | 760 |
| 100 | 600 |
| 100 | 500 |
| 100 | 400 |
| 100 | 300 |
| 100 | 200 |

### Reaction Conditions—Contact Time

Contact time of the reactants with the catalyst may range from about 0.5 seconds, about 1 second, about 5 seconds, about 10 seconds, to about 20 seconds, about 30 seconds, about 60 seconds, or about 120 seconds, or any range using any of the foregoing values as endpoints, such as from about 0.5 seconds to about 120 seconds, about 1 second to about 60 seconds, about 5 seconds to about 30 seconds, or about 10 seconds to about 20 seconds. However, longer or shorter times can be used. Specific examples of additional suitable ranges are set forth below in Table 21.

TABLE 21
Reaction Contact Time - Step (iii) Isomerization Reaction

| From (seconds) | To (seconds) |
|---|---|
| 0.5 | 120 |
| 0.5 | 60 |
| 0.5 | 30 |
| 0.5 | 20 |
| 1 | 120 |
| 1 | 60 |
| 1 | 30 |
| 1 | 20 |
| 5 | 120 |
| 5 | 60 |
| 5 | 30 |
| 5 | 20 |

### Reaction Conditions—Environment

The reaction may also be conducted in an inert atmosphere substantially in the absence of oxygen. For example, the amount of oxygen present during the reaction may be less than 15 wt. %, less than 10 wt. %, less than 5 wt. %, or less than 1 wt. %, or any range using any two of the foregoing values as endpoints, such as from about 1 wt. % to about 15 wt. %, or about 5 wt. % to about 10 wt. %, based on a total weight of the reactants in the reactor.

The reaction may also be conducted substantially in the absence of water. For example, the amount of water present during the reaction may be less than 5 wt. %, less than 1 wt. %, less than 0.5 wt. %, or less than 0.05 wt. %, or any range using any two of the foregoing values as endpoints, such as 0.05 wt. % to about 5 wt. %, or about 0.5 wt. % to about 1 wt. %, based on a total weight of the reactants in the reactor.

### EXAMPLES

### Example 1

Catalyst Conditioning—4 wt. % Pd/C with Varying Temperature

This example shows that catalyst conditioning at higher temperatures increases total selectivity to desired products in the process of R-113 conversion to R-143.

The experimental apparatus used for this example is generally described in connection with FIG. 1, and includes a feed system containing gas flow controllers for N₂ and H₂ and a Micromotion mass flow meter connected to a research control valve (RCV) controlling the organic flow rate. The reactor 112 consists of a one-inch SS tube packed with the catalyst. A thermocouple is inserted into the middle of the catalyst bed to read the operating temperature. The pressure control system consists of a RCV which is controlling the pressure by getting feedback from the pressure transducer placed after the reactor. For GC analysis, samples are taken after the reactor 112 in stream 120 using a sample bag filled with 50 ml of water to capture HCl and HF. Before the GC analysis, the sample bag is heated at 60° C. for one hour to assure that all the organic content is in the gas phase. Then, a sample is taken using a syringe and injected into the GC instrument for analysis.

Referring again to FIG. 1, streams 102, 104 and 106 may optionally be connected to a KOH scrubber which is coupled to vent. Further, after stream 120 exits the reactor 112 and as the reaction proceeds, the products may optionally be fed into a holding tank which is immersed in a bath of dry ice or a dry ice and acetone mixture. The dry ice or dry ice and acetone mixture bath is configured to hold the temperature of holding tank at a reduced temperature below ambient, such as about −87° C. The holding tank is optionally coupled to a buffer knock-out tank to prevent potential backflow of KOH solution, which is also coupled to KOH scrubber having a vent that opens to the atmosphere.

Table 22 shows the mole percentages of the product stream after the reactor determined using GC-FID analysis. For the results presented in Table 22, two samples at each temperature were collected (one sample every two hours) and then the temperature was changed to the next target. The results are presented in the order reactions were performed. The BET surface area for 4% Pd/C supported catalyst used in Example 1 was 1661.6 m²/g.

Since the substrate conversion was 100%, mole percentages are equal to selectivity.

As shown in Table 22, initially the total selectivity to undesired products were 13.54% and 16.15% at 231° C. and 250° C., respectively. After running the reaction at 280° C. for five hours, the total selectivity to undesired products was 8.95% at 231° C. and 9.27% at 250° C. The total selectivity to undesired products decreased by 4.69% at 231° C. and 6.88% at 250° C. upon five hours of pre-conditioning at 280° C. In Table 22 below, the organic feed rate was 10 g/h, H₂ feed rate was 150 ml/min, catalyst volume was 50 ml, and pressure was 45 psig.

TABLE 22
The mole composition after a reactor packed with 4% Pd/C and a feed containing >99.9% R-113

| | mole % (desired) | | | | | | mole % (undesired) | | | | | | |
|---|---|---|---|---|---|---|---|---|---|---|---|---|---|
| Temp | R- | R- | R- | R- | R- | | R- | R- | R- | R- | R-142 | | |
| (° C.) | 143 | 133b | 133 | 123a | 113 | total | 143a | 170 | 152a | 160 | isomers | Other* | total |
| 231 | 70.01 | 8.84 | 5.10 | 2.51 | 0.00 | 86.46 | 0.50 | 4.71 | 4.84 | 1.02 | 1.06 | 1.41 | 13.54 |
| 250 | 71.20 | 9.36 | 3.04 | 0.25 | 0.00 | 83.85 | 0.56 | 7.12 | 5.24 | 0.63 | 1.46 | 1.14 | 16.15 |
| 280 | 77.80 | 5.28 | 0.78 | 0.07 | 0.00 | 83.93 | 0.35 | 7.49 | 5.18 | 0.28 | 1.81 | 0.96 | 16.07 |
| 231 | 69.48 | 13.86 | 3.69 | 4.02 | 0.00 | 91.05 | 0.26 | 2.50 | 3.46 | 0.97 | 0.91 | 0.85 | 8.95 |
| 250 | 76.42 | 11.15 | 2.61 | 0.55 | 0.00 | 90.72 | 0.22 | 2.78 | 3.11 | 0.54 | 1.57 | 1.05 | 9.27 |

*Other includes R-1132a, R-1123, R-161, R-152, R-151a, and unknowns.

### Example 2

Catalyst Conditioning—1 wt. % and 4 wt. % Pd/C with Varying Temperature

This example shows that catalyst conditioning at higher temperatures increases the total selectivity to desired products in the process of R-113 conversion to R-143. The experimental conditions were similar to Example 1, but a feed containing 17±2% R-123a and 83±2% R-113. R-123a was used which is considered a recyclable intermediate in the process of R-143 production from R-113.

The product compositions were determined using GC-FID analysis of the product stream after the reactor. For the results presented in Table 23, two samples at each temperature were collected (one sample every two hours) and then the temperature was changed to the next target. The results for each catalyst are presented in the order reactions were performed. The initial temperature was repeated at the end to evaluate catalyst changes during the reaction. The BET surface area for 4% Pd/C supported catalyst used in Example 2 was 1661.6 m²/g. The BET surface area for 1% Pd/C supported catalyst used in Example 2 was 1042.2 m²/g.

As Table 23 shows, for both catalysts, when the catalyst bed temperature was returned to its initial temperature after 5 hours of reaction at 280° C., an improvement in selectivity toward the R-143 target product as well as a decrease in selectivity to undesired byproducts was observed. In Table 23, the organic feed rate was 10 g/h, H₂ feed rate was 150 ml/min, and catalyst volume was 50 ml, and pressure was 45 psig.

TABLE 23
Product compositions after the reactor for a feed containing 17 ±
2% R-123a and 83 ± 2% R-113 using 1% and 4% Pd/C catalysts

| | | mole % (desired) | | | | | | mole % (undesired) | | | | | | |
|---|---|---|---|---|---|---|---|---|---|---|---|---|---|---|
| | Temp | R- | R- | R- | R- | R- | | R- | R- | R- | R- | R- | | |
| Cat. | (° C.) | 143 | 133b | 133 | 123a | 113 | total | 143a | 170 | 152a | 160 | 142isomers | Other * | total |
| 4% | 206 | 56.44 | 10.36 | 5.77 | 19.21 | 0.00 | 91.78 | 0.15 | 1.21 | 5.08 | 0.52 | 0.39 | 0.87 | 8.22 |
| Pd/C | 230 | 66.35 | 13.43 | 6.34 | 5.19 | 0.00 | 91.31 | 0.14 | 1.86 | 4.40 | 0.45 | 0.77 | 1.07 | 8.69 |
| | 250 | 71.36 | 12.99 | 5.09 | 0.15 | 0.00 | 89.59 | 0.17 | 3.40 | 4.25 | 0.41 | 1.20 | 0.98 | 10.41 |
| | 280 | 78.44 | 5.93 | 1.47 | 0.03 | 0.01 | 85.88 | 0.20 | 6.69 | 4.05 | 0.30 | 1.72 | 1.16 | 14.12 |
| | 206 | 56.70 | 13.75 | 2.88 | 21.28 | 0.02 | 94.63 | 0.09 | 0.59 | 3.21 | 0.33 | 0.37 | 0.78 | 5.37 |
| 1% | 230 | 47.44 | 9.09 | 1.45 | 25.85 | 0.15 | 83.98 | 0.83 | 1.86 | 11.23 | 0.21 | 0.59 | 1.30 | 16.02 |
| Pd/C | 250 | 56.64 | 10.74 | 2.46 | 13.31 | 0.01 | 83.16 | 0.89 | 2.02 | 10.81 | 0.28 | 1.44 | 1.40 | 16.84 |
| | 280 | 63.65 | 12.70 | 3.37 | 1.54 | 0.04 | 81.30 | 1.02 | 2.80 | 10.02 | 0.40 | 2.77 | 1.69 | 18.70 |
| | 230 | 54.66 | 10.96 | 1.27 | 23.23 | 0.00 | 90.12 | 0.62 | 0.77 | 6.65 | 0.10 | 0.52 | 1.22 | 9.88 |

* Other includes R-1132a, R-1123, R-161, R-152, R-151a, and unknowns.

### Example 3

Catalyst Conditioning—4 wt. % Pd/C with Varying Temperature, Extended Duration

This example shows catalyst conditioning with time increases total selectivity to desired products in the process of R-113 conversion to R-143. The experimental conditions were similar to Example 1. After the experiments presented in Example 1, operation was continued at 250° C. for up to 500 hours (from the start of the run) without changing conditions. Samples were collected every four hours with the same procedure described in Example 1. The BET surface area for 4% Pd/C supported catalyst used in Example 3 was 1661.6 m²/g.

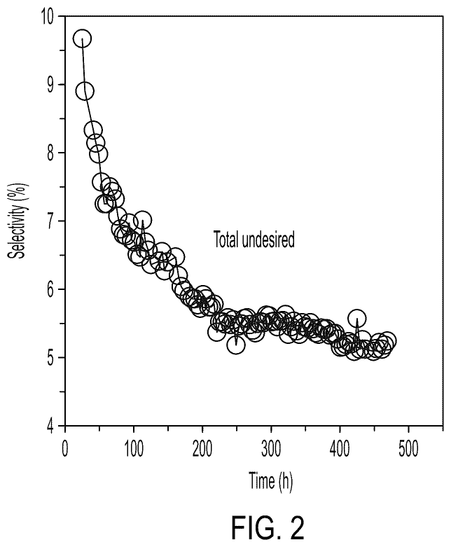

FIG. 2 shows the total selectivity to undesired by-products as a function of time for the 4% Pd/C catalyst at 250±1° C. following pre-conditioning with temperature, as presented in Example 1. The feed was >99.9% pure R-113, the organic feed rate was 10 g/h, H₂ feed rate was 150 ml/min, catalyst volume was 50 ml, and pressure was 45 psig.

Table 24 shows the results averaged over every 50 hours. Our results indicated that selectivity to desired products improves over time. The largest improvement was obtained in the first 200 hours. In Table 24, the organic feed rate was 10 g/h, H₂ feed rate was 150 ml/min, catalyst volume was 50 ml, and pressure was 45 psig.

TABLE 24
The mole composition after a reactor packed with 4% Pd/C and a feed containing >99.9% R-113

| | mole % (desired) | | | | | | mole % (undesired) | | | | | | |
|---|---|---|---|---|---|---|---|---|---|---|---|---|---|
| Time | R- | R- | R- | R- | R- | | R- | R- | R- | R- | R-142 | | |
| (h) | 143 | 133b | 133 | 123a | 113 | total | 143a | 170 | 152a | 160 | isomers | Other* | total |
| 25-50 | 75.80 | 12.41 | 2.50 | 0.69 | 0.00 | 91.40 | 0.21 | 2.51 | 3.08 | 0.54 | 1.39 | 0.87 | 8.60 |
| 50-100 | 76.08 | 13.34 | 2.33 | 1.15 | 0.00 | 92.91 | 0.15 | 1.77 | 2.55 | 0.50 | 1.38 | 0.75 | 7.10 |
| 100-150 | 74.20 | 15.28 | 2.22 | 1.76 | 0.00 | 93.46 | 0.14 | 1.54 | 2.42 | 0.52 | 1.26 | 0.66 | 6.54 |
| 150-200 | 73.12 | 16.45 | 2.19 | 2.23 | 0.00 | 93.99 | 0.13 | 1.29 | 2.22 | 0.48 | 1.23 | 0.66 | 6.01 |
| 200-250 | 73.19 | 16.57 | 2.17 | 2.47 | 0.00 | 94.40 | 0.11 | 1.11 | 2.06 | 0.43 | 1.22 | 0.67 | 5.60 |
| 250-300 | 72.25 | 17.39 | 2.16 | 2.68 | 0.00 | 94.48 | 0.10 | 1.07 | 2.02 | 0.42 | 1.20 | 0.71 | 5.52 |
| 300-350 | 71.40 | 18.15 | 2.14 | 2.78 | 0.00 | 94.47 | 0.11 | 1.05 | 2.02 | 0.42 | 1.20 | 0.73 | 5.53 |
| 350-400 | 71.53 | 18.12 | 2.10 | 2.85 | 0.00 | 94.60 | 0.11 | 0.99 | 1.96 | 0.41 | 1.21 | 0.72 | 5.40 |
| 400-450 | 71.96 | 17.80 | 2.07 | 2.94 | 0.00 | 94.77 | 0.10 | 0.91 | 1.90 | 0.40 | 1.20 | 0.72 | 5.23 |
| 450-500 | 70.76 | 18.78 | 2.06 | 3.19 | 0.00 | 94.79 | 0.11 | 0.94 | 1.95 | 0.41 | 1.14 | 0.66 | 5.21 |

*Other includes R-1132a, R-1123, R-161, R-152, R-151a, and unknowns.

### Example 4

### Reactant Dilution

Example 4 demonstrates feed dilution with an inert gas such as nitrogen improves the selectivity to R-143 in the process of R-113 conversion to R-143. Reactant dilution according to this example may also be used with each of Examples 1-3 to obtain similar results. The BET surface area for 4% Pd/C supported catalyst used in Example 4 was 1661.6 m²/g.

Table 25 shows the GC-FID area percentages (mole percentage) of the product stream after the reactor. For the results presented in Table 25, 5-6 samples at each condition were collected (one sample every four hours) and the presented data are average of 5-6 data points. Note that R-113 conversion was 100% under all the conditions presented in this example, and the selectivity to desired products (R-143, R-133b, R-133, and R-123a) was larger than 93%.

It appears that addition of N₂ to the feed increases selectivity to the R-143 final product and decreases selectivity to undesired byproducts. In Table 25, the H₂ flow rate was 150 ml/min and organic flow rate was 10 g/h.

TABLE 25
Effect of reactant dilution with nitrogen in the process
of R-113 conversion to R-143 using the 4% Pd/C catalyst

| | | | mole % (desired) | | | | | mole % (undesired) | | | | | |
|---|---|---|---|---|---|---|---|---|---|---|---|---|---|
| Temp | P | N₂ | R- | R- | R- | R- | | R- | R- | R-142 | R- | | |
| ° C. | psig | (ml/min) | 143 | 133b | 133 | 123a | Total | 170 | 152a | isomers | 160 | Other* | Total |
| 251 | 45 | 0 | 70.89 | 18.65 | 2.04 | 3.24 | 94.82 | 0.92 | 1.95 | 1.15 | 0.41 | 0.75 | 5.18 |
| 251 | 45 | 28 | 71.93 | 17.76 | 1.92 | 3.29 | 94.90 | 0.86 | 1.98 | 1.07 | 0.40 | 0.79 | 5.10 |
| 261 | 70 | 0 | 72.10 | 17.82 | 2.01 | 1.15 | 93.08 | 1.67 | 1.97 | 1.81 | 0.56 | 0.91 | 6.92 |
| 261 | 70 | 50 | 73.61 | 16.68 | 1.82 | 1.22 | 93.33 | 1.62 | 1.94 | 1.64 | 0.58 | 0.89 | 6.67 |
| 261 | 70 | 100 | 74.10 | 16.38 | 1.74 | 1.37 | 93.58 | 1.49 | 2.02 | 1.42 | 0.56 | 0.92 | 6.41 |

*Other includes R-1132a, R-143a, R-1123, R-161, R-152, R-151a, and unknowns.

### Example 5

### Catalyst Conditioning and Reactant Dilution Using 1% Pt/C and 4% Pt/C Catalyst

Examples 1˜4 are repeated using 1% Pt/C and 4% Pt/C catalysts using the same process flow and under the same reaction conditions. Similar results are obtained.

### Example 6

### Formation of HFO-1132E

The desired product (1,1,2-trifluoroethane (HFC-143)) produced in Examples 1-5 is further reacted with a catalyst to produced HFO-1132E using Steps (ii) and (iii), as described above.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances that fall within the scope of the appended claims.

### Aspects

Aspect 1 is a method for producing 1,1,2-trifluoroethane (HFC-143), comprising: reacting 1,1,2-trichloro-1,2,2-trifluoroethane (CFC-113) with hydrogen in the presence of a catalyst at a first temperature from about 250° C. to about 350° C. for at least 5 hours to produce 1,1,2-trifluoroethane (HFC-143); and continuing reacting 1,1,2-trichloro-1,2,2-trifluoroethane (CFC-113) with hydrogen in the presence of the catalyst at a second temperature from about 200° C. to about 275° C. and lower than the first temperature to produce a product composition comprising 1,1,2-trifluoroethane (HFC-143).
Aspect 2 is the method of Aspect 1, wherein the first reacting step is carried out at a temperature from at least about 270° C. to at least about 300° C.
Aspect 3 the method of Aspect 1 or Aspect 2, wherein the product composition further comprises at least one of 1-chloro-1,1,2-trifluoroethane (HCFC-133b), 1-chloro-1,2,2-trifluoroethane (HCFC-133) and 1,2-dichloro-1,1,2-trifluoroethane (HCFC-123a).
Aspect 4 is the method of Aspect 3, wherein the product composition comprises a combined total amount of 80 mol % to 99.9 mol % of 1,1,2-trifluoroethane (HFC-143), 1-chloro-1,1,2-trifluoroethane (HCFC-133b), 1-chloro-1,2,2-trifluoroethane (HCFC-133) and 1,2-dichloro-1,1,2-trifluoroethane (HCFC-123a) based on total moles of organic components in the composition.
Aspect 5 is the method of Aspect 4, wherein the product composition comprises: 65 mol % to 99.97 mol % 1,1,2-trifluoroethane (HFC-143); 0.01 mol % to 20 mol % 1-chloro-1,1,2-trifluoroethane (HCFC-133b); 0.01 mol % to 5 mol % 1-chloro-1,2,2-trifluoroethane (HCFC-133); and 0.01 mol % to 10 mol % 1,2-dichloro-1,1,2-trifluoroethane (HCFC-123a), based on the combined total moles of the HFC-143, HCFC-133b, HCFC-133, and HCFC-123a in the product composition.
Aspect 6 is the method of any one of Aspects 1-5, wherein the product composition further comprises at least one of 1,1,1-trifluoroethane (HFC-143a), ethane (HC-170), chloroethane (HCC-160), 1,1-difluoroethane (HFC-152a), and HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)).
Aspect 7 is the method of Aspect 6, wherein the product composition comprises: 55 mol % to 99.96 mol % 1,1,2-trifluoroethane (HFC-143); and a combined total of 0.01 mol % to 10 mol % of 1,1,1-trifluoroethane (HFC-143a), ethane (HC-170), chloroethane (HCC-160), 1,1-difluoroethane (HFC-152a) and HCFC-142 isomers comprising 1-chloro-1,2-difluoroethane (HCFC-142a), 1-chloro-2,2-difluoroethane (HCFC-142), or 1-chloro-1,1-difluoroethane (HCFC-142b) based on the combined total moles of the HFC-143, HCFC-133b, HCFC-133, HCFC-123a, HFC-143a, HC-170, HCC-160, HFC-152a, and HCFC-142 isomers in the product composition.
Aspect 8 is the method of Aspect 6, wherein the product composition comprises: 50 mol % to 99.96 mol % 1,1,2-trifluoroethane (HFC-143); and at least one of 0.01 mol % to 2 mol % 1,1,1-trifluoroethane (HFC-143a); 0.01 mol % to 10 mol % ethane (HC-170); 0.01 mol % to 3 mol % of chloroethane (HCC-160); 0.01 mol % to 15 mol % 1,1-difluoroethane (HFC-152a); and 0.01 mol % to 5 mol % HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), based on combined total moles of the HFC-143, HCFC-133b, HCFC-133, HCFC-123a, HFC-143a, HC-170, HCC-160, HFC-152a, and HCFC-142 isomers in the product composition.
Aspect 9 is the method of Aspect 6, wherein the product composition comprises: 55 mol % to 99.96 mol % 1,1,2-trifluoroethane (HFC-143); and at least one of 0.01 mol % to 1 mol % 1,1,1-trifluoroethane (HFC-143a); 0.01 mol % to 5 mol % ethane (HC-170); 0.01 mol % to 1 mol % of chloroethane (HCC-160); 0.01 mol % to 10 mol % 1,1-difluoroethane (HFC-152a); and 0.01 mol % to 2 mol % HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), based on combined total moles of the HFC-143, HCFC-133b, HCFC-133, HCFC-123a, HFC-143a, HC-170, HCC-160, HFC-152a, and HCFC-142 isomers in the product composition.
Aspect 10 is the method of any one of Aspects 1-9, wherein the first reacting step or the second reacting step is carried out at a pressure from about 10 to about 200 psig.
Aspect 11 is the method of any one of Aspects 1-10, further comprising diluting the 1,1,2-trichloro-1,2,2-trifluoroethane (CFC-113) with an inert gas.
Aspect 12 is the method of Aspect 11, wherein a percentage of a volumetric flow rate of the inert gas relative to a total volumetric flow rate of a reaction feed of the hydrogenation step is from about 5% to about 50%.
Aspect 13 is the method of any one of Aspects 1-12, wherein the catalyst is palladium metal supported on a carbon support.
Aspect 14 is the method of any one of Aspects 1-13, wherein the catalyst comprises from about 1 wt. % to about 5 wt. % of palladium metal supported on a carbon support, based on a total weight of the catalyst and support.
Aspect 15 is the method of Aspect 14, wherein the catalyst comprises about 4 wt. % of palladium metal supported on a carbon support, based on a total weight of the catalyst and support.
Aspect 16 is the method of any one of Aspects 1-15, further comprising reacting the product composition with a catalyst to produce trans-1,2-difluoroethylene (HFO-1132E).
Aspect 17 is a method for producing 1,1,2-trifluoroethane (HFC-143), comprising reacting 1,1,2-trichloro-1,2,2-trifluoroethane (CFC-113) with hydrogen in the presence of a catalyst and an inert diluent gas at a temperature of from about 250° C. to about 400° C. to produce a product composition comprising 1,1,2-trifluoroethane (HFC-143).
Aspect 18 is the method of Aspect 17, wherein a percentage of a volumetric flow rate of the inert gas relative to a total volumetric flow rate of a reaction feed of the hydrogenation step is from about 5% to about 50%.
Aspect 19 is the method of Aspect 17, wherein a percentage of a volumetric flow rate of the inert gas relative to a total volumetric flow rate of a reaction feed of the hydrogenation step is from about 20% to about 40%.
Aspect 20 is the method of any one of Aspects 17-19, wherein the catalyst comprises about 4 wt. % of palladium metal supported on a carbon support, based on a total weight of the catalyst and support.
Aspect 21 is the method of any one of Aspects 17-20, wherein the product composition comprises 70 mol % to 99.99 mol % 1,1,2-trifluoroethane (HFC-143); and a combined total of 0.01 mol % to 30 mol % of 1,1,1-trifluoroethane (HFC-143a), ethane (HC-170), chloroethane (HCC-160), 1,1-difluoroethane (HFC-152a) and HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), based on combined total moles of the HFC-143, HFC-143a, HC-170, HCC-160, HFC-152a, and HCFC-142 isomers in the product composition.
Aspect 22 is the method of any one of Aspects 17-21, wherein the product composition comprises a combined total of 80 mol % to 99.9 mol % of 1,1,2-trifluoroethane (HFC-143), 1-chloro-1,1,2-trifluoroethane (HCFC-133b), 1-chloro-1,2,2-trifluoroethane (HCFC-133) and 1,2-dichloro-1,1,2-trifluoroethane (HCFC-123a) based on total moles of organic components in the product composition.
Aspect 23 is the method of any one of Aspects 17-22, wherein the product composition comprises: 70 mol % to 99.99 mol % 1,1,2-trifluoroethane (HFC-143); and at least one of 0.01 mol % to 2 mol % ethane (HC-170); 0.01 mol % to 1 mol % of chloroethane (HCC-160); 0.01 mol % to 2 mol % 1,1-difluoroethane (HFC-152a); and 0.01 mol % to 2 mol % HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), based on total moles of organic components in the composition.
Aspect 24 is the method of any one of Aspects 17-23, further comprising reacting the product composition with a catalyst to produce trans-1,2-difluoroethylene (HFO-1132E).
Aspect 25 is the method of any one of Aspects 1-16, wherein the catalyst comprises from about 0.5 wt. % to about 10 wt. % of palladium metal supported on a carbon support, based on a total weight of the catalyst and support, and wherein the first hydrogenating step is carried out at a temperature of from about 100° C. to about 350° C.
Aspect 26 is the method of any one of Aspects 1-16, wherein the catalyst comprises from about 0.5 wt. % to about 10 wt. % of palladium metal supported on a carbon support, based on a total weight of the catalyst and support, and wherein the first hydrogenating step is carried out at a temperature of from about 200° C. to about 300° C.
Aspect 27 is the method of any one of Aspects 1-16, wherein the catalyst comprises from about 0.5 wt. % to about 10 wt. % of palladium metal supported on a carbon support, based on a total weight of the catalyst and support, and wherein the first hydrogenating step is carried out at a temperature of from about 230° C. to about 290° C.
Aspect 28 is the method of any one of Aspects 1-16, wherein the catalyst comprises from about 0.5 wt. % to about 10 wt. % of palladium metal supported on a carbon support, based on a total weight of the catalyst and support, and wherein the first hydrogenating step is carried out at a temperature of from about 250° C. to about 280° C.
Aspect 29 is the method of any one of Aspects 1-16, wherein the catalyst comprises from about 1 wt. % to about 5 wt. % of palladium metal supported on a carbon support, based on a total weight of the catalyst and support, and wherein the first hydrogenating step is carried out at a temperature of from about 100° C. to about 350° C.
Aspect 30 is the method of any one of Aspects 1-16, wherein the catalyst comprises from about 1 wt. % to about 5 wt. % of palladium metal supported on a carbon support, based on a total weight of the catalyst and support, and wherein the first hydrogenating step is carried out at a temperature of from about 200° C. to about 300° C.
Aspect 31 is the method of any one of Aspects 1-16, wherein the catalyst comprises from about 1 wt. % to about 5 wt. % of palladium metal supported on a carbon support, based on a total weight of the catalyst and support, and wherein the first hydrogenating step is carried out at a temperature of from about 230° C. to about 290° C.
Aspect 32 is the method of any one of Aspects 1-16, wherein the catalyst comprises from about 1 wt. % to about 5 wt. % of palladium metal supported on a carbon support, based on a total weight of the catalyst and support, and wherein the first hydrogenating step is carried out at a temperature of from about 250° C. to about 280° C.
Aspect 33 is the method of any one of Aspects 17-24, wherein the catalyst comprises from about 0.5 wt. % to about 10 wt. % of palladium metal supported on a carbon support, based on a total weight of the catalyst and support, and wherein the first hydrogenating step is carried out at a temperature of from about 100° C. to about 350° C.
Aspect 34 is the method of any one of Aspects 17-24, wherein the catalyst comprises from about 0.5 wt. % to about 10 wt. % of palladium metal supported on a carbon support, based on a total weight of the catalyst and support, and wherein the first hydrogenating step is carried out at a temperature of from about 200° C. to about 300° C.
Aspect 35 is the method of any one of Aspects 17-24, wherein the catalyst comprises from about 0.5 wt. % to about 10 wt. % of palladium metal supported on a carbon support, based on a total weight of the catalyst and support, and wherein the first hydrogenating step is carried out at a temperature of from about 230° C. to about 290° C.
Aspect 36 is the method of any one of Aspects 17-24, wherein the catalyst comprises from about 0.5 wt. % to about 10 wt. % of palladium metal supported on a carbon support, based on a total weight of the catalyst and support, and wherein the first hydrogenating step is carried out at a temperature of from about 250° C. to about 280° C.
Aspect 37 is the method of any one of Aspects 17-24, wherein the catalyst comprises from about 1 wt. % to about 5 wt. % of palladium metal supported on a carbon support, based on a total weight of the catalyst and support, and wherein the first hydrogenating step is carried out at a temperature of from about 100° C. to about 350° C.
Aspect 38 is the method of any one of Aspects 17-24, wherein the catalyst comprises from about 1 wt. % to about 5 wt. % of palladium metal supported on a carbon support, based on a total weight of the catalyst and support, and wherein the first hydrogenating step is carried out at a temperature of from about 200° C. to about 300° C.
Aspect 39 is the method of any one of Aspects 17-24, wherein the catalyst comprises from about 1 wt. % to about 5 wt. % of palladium metal supported on a carbon support, based on a total weight of the catalyst and support, and wherein the hydrogenating step is carried out at a temperature of from about 230° C. to about 290° C.
Aspect 40 is the method of any one of Aspects 17-24, wherein the catalyst comprises from about 1 wt. % to about 5 wt. % of palladium metal supported on a carbon support, based on a total weight of the catalyst and support, and wherein the first hydrogenating step is carried out at a temperature of from about 250° C. to about 280° C.
Aspect 41 is a product composition comprising: 80 mol % to 99.99 mol % 1,1,2-trifluoroethane (HFC-143); and at least one of 0.01 mol % to 20 mol % 1-chloro-1,1,2-trifluoroethane (HCFC-133b); 0.01 mol % to 5 mol % 1-chloro-1,2,2-trifluoroethane (HCFC-133); and 0.01 mol % to 10 mol % 1,2-dichloro-1,1,2-trifluoroethane (HCFC-123a), based on combined total moles of the HFC-143, HCFC-133b, HCFC-133, and HCFC-123a in the product composition.
Aspect 42 is the product composition of Aspect 41, comprising 90 mol % to 99.99 mol % 1,1,2-trifluoroethane (HFC-143) and further comprising at least one of: 0.01 mol % to 1 mol % 1,1,1-trifluoroethane (HFC-143a); 0.01 mol % to 5 mol % ethane (HC-170); 0.01 mol % to 1 mol % of chloroethane (HCC-160); 0.01 mol % to 10 mol % 1,1-difluoroethane (HFC-152a); and 0.01 mol % to 2 mol % HCFC-142 isomers comprising 1-chloro-1,2-difluoroethane (HCFC-142a), 1-chloro-2,2-difluoroethane (HCFC-142), or 1-chloro-1,1-difluoroethane (HCFC-142b), based on combined total moles of the HFC-143, HCFC-133b, HCFC-133, HCFC-123a, HFC-143a, HC-170, HCC-160, HFC-152a, and HCFC-142 isomers in the product composition.
Aspect 43 is a product composition comprising: 80 mol % to 99.99 mol % 1,1,2-trifluoroethane (HFC-143); and at least one of 0.01 mol % to 20 mol % 1-chloro-1,1,2-trifluoroethane (HCFC-133b); 0.01 mol % to 3 mol % 1-chloro-1,2,2-trifluoroethane (HCFC-133); and 0.01 mol % to 4 mol % 1,2-dichloro-1,1,2-trifluoroethane (HCFC-123a), combined total moles of the HFC-143, HCFC-133b, HCFC-133, and HCFC-123a in the product composition.

Aspect 44 is the product composition of Aspect 43, comprising 78 mol % to 99.98 mol % 1,1,2-trifluoroethane (HFC-143); and further comprising at least one of: 0.01 mol % to 1 mol % 1,1,1-trifluoroethane (HFC-143a); 0.01 mol % to 2 mol % ethane (HC-170); 0.01 mol % to 1 mol % of chloroethane (HCC-160); 0.01 mol % to 2 mol % 1,1-difluoroethane (HFC-152a); and 0.01 mol % to 2 mol % HCFC-142 isomers comprising 1-chloro-1,2-difluoroethane (HCFC-142a), 1-chloro-2,2-difluoroethane (HCFC-142), or 1-chloro-1,1-difluoroethane (HCFC-142b), combined total moles of the HFC-143, HCFC-133b, HCFC-133, HCFC-123a, HFC-143a, HC-170, HCC-160, HFC-152a, and HCFC-142 isomers in the product composition.

What is claimed is:

1. A method for producing 1,1,2-trifluoroethane (HFC-143), comprising:
reacting 1,1,2-trichloro-1,2,2-trifluoroethane (CFC-113) with hydrogen in the presence of a catalyst at a first temperature from about 250° C. to about 350° C. for at least 5 hours to produce 1,1,2-trifluoroethane (HFC-143); and
continuing reacting 1,1,2-trichloro-1,2,2-trifluoroethane (CFC-113) with hydrogen in the presence of the catalyst at a second temperature from about 200° C. to about 275° C. and lower than the first temperature to produce a product composition comprising 1,1,2-trifluoroethane (HFC-143).

2. The method of claim 1, wherein the first reacting step is carried out at a temperature from at least about 270° C. to at least about 300° C.

3. The method of claim 1, wherein the product composition further comprises at least one of 1-chloro-1,1,2-trifluoroethane (HCFC-133b), 1-chloro-1,2,2-trifluoroethane (HCFC-133) and 1,2-dichloro-1,1,2-trifluoroethane (HCFC-123a).

4. The method of claim 3, wherein the product composition comprises:
a combined total amount of 80 mol % to 99.9 mol % of 1,1,2-trifluoroethane (HFC-143), 1-chloro-1,1,2-trifluoroethane (HCFC-133b), 1-chloro-1,2,2-trifluoroethane (HCFC-133) and 1,2-dichloro-1,1,2-trifluoroethane (HCFC-123a) based on total moles of organic components in the composition.

5. The method of claim 3, wherein the product composition comprises:
65 mol % to 99.97 mol % 1,1,2-trifluoroethane (HFC-143);
0.01 mol % to 20 mol % 1-chloro-1,1,2-trifluoroethane (HCFC-133b);
0.01 mol % to 5 mol % 1-chloro-1,2,2-trifluoroethane (HCFC-133); and
0.01 mol % to 10 mol % 1,2-dichloro-1,1,2-trifluoroethane (HCFC-123a), based on the combined total moles of the HFC-143, HCFC-133b, HCFC-133, and HCFC-123a in the product composition.

6. The method of claim 1, wherein the product composition further comprises at least one of 1,1,1-trifluoroethane (HFC-143a), ethane (HC-170), chloroethane (HCC-160), 1,1-difluoroethane (HFC-152a), and HCFC-142 isomers comprising 1-chloro-1,2-difluoroethane (HCFC-142a), 1-chloro-2,2-difluoroethane (HCFC-142), or 1-chloro-1,1-difluoroethane (HCFC-142b).

7. The method of claim 6, wherein the product composition comprises:
55 mol % to 99.96 mol % 1,1,2-trifluoroethane (HFC-143); and
a combined total of 0.01 mol % to 10 mol % of 1,1,1-trifluoroethane (HFC-143a), ethane (HC-170), chloroethane (HCC-160), 1,1-difluoroethane (HFC-152a) and HCFC-142 isomers comprising 1-chloro-1,2-difluoroethane (HCFC-142a), 1-chloro-2,2-difluoroethane (HCFC-142), or 1-chloro-1,1-difluoroethane (HCFC-142b) based on the combined total moles of the HFC-143, HCFC-133b, HCFC-133, HCFC-123a, HFC-143a, HC-170, HCC-160, HFC-152a, and HCFC-142 isomers in the product composition.

8. The method of claim 6, wherein the product composition comprises:
50 mol % to 99.96 mol % 1,1,2-trifluoroethane (HFC-143); and
at least one of:
0.01 mol % to 2 mol % 1,1,1-trifluoroethane (HFC-143a);
0.01 mol % to 10 mol % ethane (HC-170);
0.01 mol % to 3 mol % of chloroethane (HCC-160);
0.01 mol % to 15 mol % 1,1-difluoroethane (HFC-152a); and
0.01 mol % to 5 mol % HCFC-142 isomers comprising 1-chloro-1,2-difluoroethane (HCFC-142a), 1-chloro-2,2-difluoroethane (HCFC-142), or 1-chloro-1,1-difluoroethane (HCFC-142b), based on combined total moles of the HFC-143, HCFC-133b, HCFC-133, HCFC-123a, HFC-143a, HC-170, HCC-160, HFC-152a, and HCFC-142 isomers in the product composition.

9. The method of claim 6, wherein the product composition comprises:
55 mol % to 99.96 mol % 1,1,2-trifluoroethane (HFC-143); and
at least one of:
0.01 mol % to 1 mol % 1,1,1-trifluoroethane (HFC-143a);
0.01 mol % to 5 mol % ethane (HC-170);
0.01 mol % to 1 mol % of chloroethane (HCC-160);
0.01 mol % to 10 mol % 1,1-difluoroethane (HFC-152a); and
0.01 mol % to 2 mol % HCFC-142 isomers comprising 1-chloro-1,2-difluoroethane (HCFC-142a), 1-chloro-2,2-difluoroethane (HCFC-142), or 1-chloro-1,1-difluoroethane (HCFC-142b), based on combined total moles of the HFC-143, HCFC-133b, HCFC-133, HCFC-123a, HFC-143a, HC-170, HCC-160, HFC-152a, and HCFC-142 isomers in the product composition.

10. The method of claim 9, wherein the first reacting step or the second reacting step is carried out at a pressure from about 10 to about 200 psig.

11. The method of claim 10, wherein the catalyst is palladium metal supported on a carbon support.

12. The method of claim 11, wherein the catalyst comprises from about 1 wt. % to about 5 wt. % of palladium metal supported on a carbon support, based on a total weight of the catalyst and support.

13. The method of claim 12, further comprising reacting the product composition with a catalyst to produce trans-1,2-difluoroethylene (HFO-1132E).

14. A method for producing 1,1,2-trifluoroethane (HFC-143), comprising:
reacting 1,1,2-trichloro-1,2,2-trifluoroethane (CFC-113) with hydrogen in the presence of a catalyst and an inert diluent gas at a temperature of from about 250° C. to about 400° C. to produce a product composition comprising 1,1,2-trifluoroethane (HFC-143).

15. The method of claim 14, wherein a percentage of a volumetric flow rate of the inert gas relative to a total volumetric flow rate of a reaction feed of the hydrogenation step is from about 5% to about 50%.

16. The method of claim 14, wherein the catalyst comprises about 4 wt. % of palladium metal supported on a carbon support, based on a total weight of the catalyst and support.

17. The method of claim 16, wherein the product composition comprises:
70 mol % to 99.99 mol % 1,1,2-trifluoroethane (HFC-143); and
a combined total of 0.01 mol % to 30 mol % of 1,1,1-trifluoroethane (HFC-143a), ethane (HC-170), chloroethane (HCC-160), 1,1-difluoroethane (HFC-152a) and HCFC-142 isomers comprising 1-chloro-1,2-difluoroethane (HCFC-142a), 1-chloro-2,2-difluoroethane (HCFC-142), or 1-chloro-1,1-difluoroethane (HCFC-142b), based on combined total moles of the HFC-143, HFC-143a, HC-170, HCC-160, HFC-152a, and HCFC-142 isomers in the product composition.

18. The method of claim 17, wherein the product composition comprises:
a combined total of 80 mol % to 99.99 mol % of 1,1,2-trifluoroethane (HFC-143), 1-chloro-1,1,2-trifluoroethane (HCFC-133b), 1-chloro-1,2,2-trifluoroethane (HCFC-133) and 1,2-dichloro-1,1,2-trifluoroethane (HCFC-123a) based on total moles of organic components in the product composition.

19. The method of claim 18, wherein the product composition comprises:
70 mol % to 99.99 mol % 1,1,2-trifluoroethane (HFC-143); and
at least one of:
0.01 mol % to 2 mol % ethane (HC-170);
0.01 mol % to 1 mol % of chloroethane (HCC-160);
0.01 mol % to 2 mol % 1,1-difluoroethane (HFC-152a); and
0.01 mol % to 2 mol % HCFC-142 isomers comprising 1-chloro-1,2-difluoroethane (HCFC-142a), 1-chloro-2,2-difluoroethane (HCFC-142), or 1-chloro-1,1-difluoroethane (HCFC-142b), based on total moles of organic components in the composition.

20. The method of claim 16, further comprising reacting the product composition with a catalyst to produce trans-1,2-difluoroethylene (HFO-1132E).
